(19) United States
(12) Patent Application Publication
(10) Pub. No.: US 2023/0137198 A1
(43) Pub. Date: May 4, 2023

(54) APPROXIMATING MOTION CAPTURE OF PLURAL BODY PORTIONS USING A SINGLE IMU DEVICE

(71) Applicant: Celloscope Ltd., Tel Aviv (IL)

(72) Inventors: Yuval NAVEH, Haifa (IL); Tamir ALOUSH, Kiryat Ono (IL)

(21) Appl. No.: 17/666,180

(22) Filed: Feb. 7, 2022

Related U.S. Application Data
(60) Provisional application No. 63/272,839, filed on Oct. 28, 2021.
(60) Provisional application No. 63/305,203, filed on Jan. 31, 2022.

(51) Int. Cl. A61B 5/11; G01P 15/18; A61B 5/00

(52) U.S. Cl. CPC A61B 5/1122; A61B 5/744; A61B 5/1112; A61B 5/1126; A61B 5/7267; A61B 5/7275; G01P 15/18; A61B 2562/0219; A61B 2562/0223

(57) ABSTRACT

A system capturing motion of a moving body, including an IMU interface to receive IMU measurements from IMU/s worn on the body; and a processor to derive, from the IMU measurements, a motion capture approximation output including a trajectory, which describes a body portion’s motion during a cycle of repetitive motion, yielding a trajectory set including B body portion trajectories, wherein, to derive the trajectory set, the processor uses generative adversarial networks including one network trained to determine physical feasibility of candidate body portion trajectory/ies for body portion/s, from among multiple candidate body portion trajectories for the specific body portion; and another network trained to determine how well candidate body portion trajectory/ies fit/s the IMU measurements.

Priority is claimed from U.S. Provisional Pat. Application No. 63/272,839, “A motion capture system based on a single IMU device”, filed Oct. 28, 2021, the disclosure of which application is incorporated herein by reference.

### FIELD OF THIS DISCLOSURE

The present invention relates generally to motion capture.

### BACKGROUND FOR THIS DISCLOSURE

BVH files are described here: https://www.file-extension.info/format/bvh.

A method for generating a BVH FILE is described here www.cs.cityu.edu.hk/~howard/Teaching/CS4185-5185-2007-SemA/Group12/BVH.html

Trend analysis and anomaly detection are known methods

Also known, are methods for detecting patterns in sensor data e.g. as described here: www.researchgate.net/publication/329033123_Detecting_Irregular__Patterns__in_IoT_Streaming_ Data_for_Fall_Detection

Padding is a known operation in the art of neural networks and is described e.g. here: d21.ai/chapter_convolutional-neural-networks/padding-and-strides.html

Motion capture is known. Wikipedia (https://en.wikipedia.org/wiki/Motion_capture) enthuses that “Motion capture offers several advantages over traditional computer animation of a 3D model”. However, Wikipedia laments, motion capture suffers from various disadvantages, including:

“Specific hardware and special software programs are required to obtain and process the data.
The cost of the software, equipment and personnel required can be prohibitive for small productions.
The capture system may have specific requirements for the space it is operated in, depending on camera field of view or magnetic distortion.
When problems occur, it is easier to shoot the scene again rather than trying to manipulate the data. Only a few systems allow real time viewing of the data to decide if the take needs to be redone.
The initial results are limited to what can be performed within the capture volume without extra editing of the data.”

Wikipedia also describes “Inertial motion capture technology [in which] “motion data of the inertial sensors... often transmitted wirelessly to a computer, where the motion is recorded or viewed. Most inertial systems use inertial measurement units (IMUs) containing a combination of gyroscope, magnetometer, and accelerometer, to measure rotational rates. These rotations are translated to a skeleton in the software. Much like optical markers, the more IMU sensors, the more natural the data.....These rotations are translated to a skeleton in the software. No external cameras, emitters or markers are needed for relative motions, although they are required to give the absolute position of the user if desired.....Disadvantages include lower positional accuracy and positional drift which can compound over time....The popularity of inertial systems is rising amongst game developers,^{[10]} mainly because of the quick and easy set up resulting in a fast pipeline. A range of suits are now available from various manufacturers and base prices range from $1,000 to US$80,000.”

Motion analysis, or motion capture, which yields kinematic variables such as joint angles, is known, and is referred to for example, in the following publication: jneuroengrehab.biomedcentral.com/articles/10.1186/1743-0003-3-18 which describes a protocol including set-up of cameras, positioning of markers and providing an optoelectronic system for three-dimensional kinematic motion capture. Also, arxiv.org/pdf/1907.00837.pdf describes real-time monocular RGB based 3D motion capture to derive 3D skeletal pose data including plural skeletal joint angles.

Conventional motion capture may assume that an accelerometer and/or marker is deployed at each body part for which it is desired to measure a trajectory.

According to Wikipedia, “Video games often use motion capture to animate athletes, martial artists, and other in-game characters.... Movies use motion capture for CG effects, in some cases replacing traditional cel animation, and for completely computer-generated creatures.... The film Batman Forever (1995) used some motion capture for certain special effects....The Lord of the Rings: The Two Towers was the first feature film to utilize a real-time motion capture system. This method streamed the actions of actor Andy Serkis into the computer generated skin of Gollum/Smeagol as it was being performed.”. actors may provide motions and voices to plural digital characters. Also “Virtual reality and Augmented reality providers, such as uSens and Gestigon, allow users to interact with digital content in real time by capturing hand motions. This can be useful for training simulations, visual perception tests, or performing virtual walk-throughs in a 3D environment. Motion capture technology is frequently used in digital puppetry systems to drive computer generated characters in real-time. ”

Also “Gait analysis is one application of motion capture in clinical medicine. Techniques allow clinicians to evaluate human motion across several biomechanical factors, often while streaming this information live into analytical software. Some physical therapy clinics utilize motion capture as an objective way to quantify patient progress.”

Autodesk MotionBuilder software may be used to render a screen image.

Body tracking technology, available from Perception Neuron, may be used to map body movement onto a digital character’s motion on a screen.

Segmentation of a motion record, e.g. representing gait into strides, is described in co-pending published U.S. Pat. Application re Assessment of a User’s Gait (Application #20200289027 published Sep. 17, 2020).

Gait analysis and related technologies are described in Apple’s published U.S. Pat. Applications 20210393166 and 20210393162.

The disclosures of all publications and patent documents mentioned in the specification, and of the publications and patent documents cited therein directly or indirectly, are hereby incorporated by reference other than subject matter disclaimers or disavowals. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

### SUMMARY OF CERTAIN EMBODIMENTS

Certain embodiments seek to generate training data for machine learning, which include both gold-standard MoCap (Motion Capture) data and mobile phone data synchronized thereto, so as to train a system to estimate motion of a user based on mobile phone data provided by a mobile phone worn by the user, thereby to yield motion estimates which approximate traditional motion capture data.

Certain embodiments seek to provide motion capture including estimating trajectory/ies of at least one body part of a human end-user, using wearable sensor/s deployed at respective bodily position/s or body locations, typically but not necessarily a single sensor, which is deployed at a single bodily position or body location. Typically, the motion capture estimates a trajectory of at least one body part, where no sensor is deployed For example, a single sensor may be deployed, say, in the end-user’s pocket, and the motion capture may estimate trajectory/ies of joint/s such as elbow/s, knee/s and ankle/s, or limb portions such as, say, left forearm, even though no sensor is deployed at the elbow, knee or ankle or left forearm whose trajectory is being estimated by the motion capture process.

Certain embodiments seek to provide a system using trained adversarial neural networks to predict MoCap data for a set of body portions (e.g. joints) including plural body portions, using input arriving from what may be only a single IMU affixed to only a single body portion or to a smaller set of body portions than the set of body portions whose motions are of interest.

Certain embodiments seek to provide a system which includes a processor and memory circuitry (PMC) operatively connected to a hardware-based I/O interface. The PMC may be configured to provide processing for operating the system e.g. as further detailed herein and may comprises a processor and memory which may be accessible thereto. The processor of the PMC may be configured to execute functional modules in accordance with computer-readable instructions implemented on a non-transitory computer-readable memory comprised in the PMC. Such functional modules are referred to hereinafter as comprised in the PMC. Thus the scope of the invention, according to certain embodiments, includes any computerized system comprising processing and memory circuitry (PMC) configured to perform any operations shown and described herein.

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented, as appropriate.

It is appreciated that any reference herein to, or recitation of, an operation being performed is, e.g. if the operation is performed at least partly in software, intended to include both an embodiment where the operation is performed in its entirety by a server A, and also to include any type of “outsourcing” or “cloud” embodiments in which the operation, or portions thereof, is or are performed by a remote processor P (or several such), which may be deployed off-shore or “on a cloud”, and an output of the operation is then communicated to, e.g. over a suitable computer network, and used by, server A. Analogously, the remote processor P may not, itself, perform all of the operations, and, instead, the remote processor P itself may receive output/s of portion/s of the operation from yet another processor/s P′, may be deployed off-shore relative to P, or “on a cloud”, and so forth.

There is thus provided, in accordance with at least one embodiment of the present invention,

The present invention typically includes at least the following embodiments:
Embodiment 1. A system for capturing motion of a moving body, the system including: an IMU interface which may be configured to receive IMU measurements e.g., from at least one IMU worn on the moving body; and/or a hardware processor which may be configured to derive, e.g. from the IMU measurements, a motion capture approximation output which may include a trajectory, typically for each individual body portion from among B body portions, which may describe the individual body portion’s motion during a single repetition (aka cycle) of a repetitive motion, which may then yield a trajectory set including B body portion trajectories (aka “repetitive activity patterns”). Typically, the hardware processor uses generative adversarial networks e.g. to derive the trajectory set from the IMU measurements, the generative adversarial networks including a first network which may be trained to determine physical feasibility of, typically, at least one candidate body portion trajectory for at least one specific body portion, typically from among a multiplicity of candidate body portion trajectories for the specific body portion; and a second network which may be trained to determine how well at least one candidate body portion trajectory, e.g. from among the multiplicity of candidate body portion trajectories, fits the IMU measurements.
Embodiment 2. The system of any of the preceding embodiments wherein the B body portion trajectories are represented in BVH format.
Embodiment 3. The system of any of the preceding embodiments wherein at least one IMU comprises a single IMU deployed at a single body location.
Embodiment 4. The system of any of the preceding embodiments wherein at least one IMU comprises plural IMU’s deployed at plural respective body locations.
Embodiment 5. The system of any of the preceding embodiments wherein the first network includes a discriminative network trained to determine how feasible is/are candidate body portion trajectory/ies from among a multiplicity of candidate body portion trajectories, and wherein the second network includes a generative network.
Embodiment 6. The system of any of the preceding embodiments wherein the generative network is trained to predict body portion trajectory/ies, by identifying candidate body portion trajectory/ies which fit/s the IMU measurements, from among candidate body portion trajectory/ies considered feasible by the discriminative network.
Embodiment 7. The system of any of the preceding embodiments wherein the IMU measurements comprise acceleration measurements and orientation measurements.
Embodiment 8. The system of any of the preceding embodiments wherein the IMU comprises at least one of: an accelerometer; a gyroscope, a magnetometer.
Embodiment 9. The system of any of the preceding embodiments wherein the body part includes at least one of: a joint, a limb portion, a limb.
Embodiment 10. The system of any of the preceding embodiments and wherein the hardware processor is also configured for segmentation of IMU measurements of at least one body portion’s motion, into repetitions, aka cycles
Embodiment 11. The system of any of the preceding embodiments wherein the trajectory of at least one body portion from among B body portions, which describes that body portion’s motion during a single repetition, is agnostic to the IMU’s orientation.
Embodiment 12. The system of any of the preceding embodiments wherein the IMU is tri-axial.
Embodiment 13. The system of any of the preceding embodiments wherein training data to train at least one of the first and second networks includes IMU measurements provided by the at least one IMU during at least one time interval, wherein the IMU measurements are synchronized to gold-standard motion capture measurements made during the at least one time interval.
Embodiment 14. The system of any of the preceding embodiments wherein the motion capture approximation output, rather than motion capture data, is fed to at least one apparatus which utilizes motion capture data.
Embodiment 15. The system of any of the preceding embodiments wherein the apparatus comprises software which generates at least one animation of an avatar from motion capture data representing a motion, performed by a human, which the avatar is to perform.
Embodiment 16. The system of any of the preceding embodiments wherein the apparatus comprises a device for tracking at-risk persons including using motion capture data as a vital sign indicative of wellbeing of the at-risk person.
Embodiment 17. The system of any of the preceding embodiments wherein the apparatus comprises an ambulation rehabilitation tool which uses motion capture data to track a patient’s ambulation between the patient’s physical meetings with a clinician.
Embodiment 18. The system of any of the preceding embodiments wherein the apparatus comprises trend analysis and/or anomaly detection and/or pattern detection software which detects trends and/or anomalies and/or patterns in motion capture data.
Embodiment 19. The system of any of the preceding embodiments wherein the IMU is co-located with a first moving body portion having a first trajectory of motion, and wherein the system uses readings generated by the IMU to approximate motion capture data regarding motion of at least one second moving body portion having a second trajectory of motion which differs from the first trajectory of motion.

Typically, the second moving body portion can only move along the second trajectory if the first moving body portion moves along the first trajectory because the second body portion’s motions are not independent of the first body portion’s motions.

Embodiment 20. The system of any of the preceding embodiments wherein the second portion’s motion affects the first body portion’s motion.

Embodiment 21. The system of any of the preceding embodiments wherein the second portion’s motion is affected by the first body portion’s motion.

It is appreciated that body portions affect one another as a subject e.g. human moves because a subject’s movement (such as walking, running, etc.)is typically a complex activity that involves balance and synchronization between various body parts.

Embodiment 22. A method for capturing motion of a moving body, the method including:
providing an IMU interface configured to receive IMU measurements from at least one IMU worn on the moving body; and
deriving, from the IMU measurements, using a hardware processor, a motion capture approximation output including a trajectory, for each individual body portion from among B body portions, which describes the individual body portion’s motion during a single repetition (aka cycle) of a repetitive motion, thereby to provide a trajectory set including B body portion trajectories (aka “repetitive activity patterns”),
wherein the hardware processor uses generative adversarial networks to derive the trajectory set from the IMU measurements, the generative adversarial networks including:
   a first network trained to determine physical feasibility of at least one candidate body portion trajectory for at least one specific body portion, from among a multiplicity of candidate body portion trajectories for the specific body portion, and
   a second network trained to determine how well at least one candidate body portion trajectory, from among the multiplicity of candidate body portion trajectories, fits the IMU measurements.

Embodiment 22. A computer program product, comprising a non-transitory tangible computer readable medium having computer readable program code embodied therein, the computer readable program code adapted to be executed to implement a method for capturing motion of a moving body, the method including:
providing an IMU interface configured to receive IMU measurements from at least one IMU worn on the moving body; and
deriving, from the IMU measurements, using a hardware processor, a motion capture approximation output including a trajectory, for each individual body portion from among B body portions, which describes the individual body portion’s motion during a single repetition (aka cycle) of a repetitive motion, thereby to provide a trajectory set including B body portion trajectories (aka “repetitive activity patterns”),
wherein the hardware processor uses generative adversarial networks to derive the trajectory set from the IMU measurements, the generative adversarial networks including:
   a first network trained to determine physical feasibility of at least one candidate body portion trajectory for at least one specific body portion, from among a multiplicity of candidate body portion trajectories for the specific body portion; and
   a second network trained to determine how well at least one candidate body portion trajectory, from among the multiplicity of candidate body portion trajectories, fits the IMU measurements.

Also provided, excluding signals, is a computer program comprising computer program code means for performing any of the methods shown and described herein when the program is run on at least one computer; and a computer program product, comprising a typically non-transitory computer-usable or -readable medium e.g. non-transitory computer -usable or -readable storage medium, typically tangible, having a computer readable program code embodied therein, the computer readable program code adapted to be executed to implement any or all of the methods shown and described herein. The operations in accordance with the teachings herein may be performed by at least one computer specially constructed for the desired purposes, or general purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display e.g. on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMs, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g. BLE) or wired (e.g. USB)), a computer program stored in memory/computer storage.

The term “process” as used above is intended to include any type of computation or manipulation or transformation of data represented as physical, e.g. electronic, phenomena which may occur or reside e.g. within registers and /or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g., via a wired or cellular telephone network or a computer network such as the Internet.

The apparatus of the present invention may include, according to certain embodiments of the invention, machine readable memory containing or otherwise storing a program of instructions which, when executed by the machine, implements all or any subset of the apparatus, methods, features and functionalities of the invention shown and described herein. Alternatively or in addition, the apparatus of the present invention may include, according to certain embodiments of the invention, a program as above which may be written in any conventional programming language, and optionally a machine for executing the program such as but not limited to a general purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of sits owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities eg. within the computing system’s registers and/or memories, and/or may be provided on-the-fly, into other data which may be similarly represented as physical quantities within the computing system’s memories, registers or other such information storage, transmission or display devices or may be provided to external factors e.g. via a suitable data network. The term “computer” should be broadly construed to cover any kind of electronic device with data processing capabilities, including, by way of non-limiting example, personal computers, servers, embedded cores, computing systems, communication devices, processors (e.g., digital signal processors (DSPs), microcontrollers, field programmable gate arrays (FPGAs), application specific integrated circuits (ASIC), etc.) and other electronic computing devices. Any reference to a computer, controller or processor is intended to include one or more hardware devices e.g., chips, which may be co-located or remote from one another. Any controller or processor may for example comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs), or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably e.g. a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface or other system illustrated or described herein. Any suitable computerized data storage, e.g., computer memory, may be used to store information received by or generated by the systems shown and described herein Functionalities shown and described herein may be divided between a server computer and a plurality of client computers. These or any other computerized components shown and described herein may communicate between themselves via a suitable computer network.

The system shown and described herein may include user interface/s e.g. as described herein which may for example include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user’s device, automated speech-to-text conversion tool, including a front-end interface portion thereof, and back-end logic interacting therewith. Thus the term user interface or “UI” as used herein, includes also the underlying logic which controls the data presented to the user e.g. by the system display, and receives and processes and/or provides to other modules herein, data entered by a user e.g. using her or his workstation/device.

### BRIEF DESCRIPTION OF THE DRAWINGS

Example embodiments are illustrated in the various drawings. Specifically:

FIGS. 1 - 3 are graphs taken from co-pending USSN 17/500,744 filed Oct. 13, 2021, the disclosure of which is incorporated herein by reference.

FIG. 4a - 7 are simplified flowchart illustrations of methods according to certain embodiments. The methods of each typically comprise all or any subset of the illustrated operations, suitably ordered e.g., as shown. It is appreciated that the method of FIG. 5 may be used to implement operation 10c and/or operation 30b, including segmentation of repetitions of similar movements, the method of FIG. 6 may be used to implement operation 30c, including reconstruction of the pattern representation, and the method of FIG. 7 may be used to implement operation 10d of FIG. 4b. The methods of FIG. 4a-6 may be used for implementing operation 10d and/or 30c, for example. It is appreciated that stages 30c-a, 30c-b and 30c-c of FIG. 6 may respectively be identical to stages 10-1-a, 10-1-b and 10-c of FIG. 7.

Certain embodiments of the present invention are illustrated in the following drawings; in the block diagrams, arrows between modules may be implemented as APIs and any suitable technology may be used for interconnecting functional components or modules illustrated herein in a suitable sequence or order e.g. via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML. According to one embodiment, one of the modules may share a secure API with another. Communication between modules may comply with any customized protocol or customized query language, or may comply with any conventional query language or protocol.

Methods and systems included in the scope of the present invention may include any subset or all of the functional blocks shown in the specifically illustrated implementations by way of example, in any suitable order e.g., as shown. Flows may include all or any subset of the illustrated operations, suitably ordered e.g., as shown. Tables herein may include all or any subset of the fields and/or records and/or cells and/or rows and/or columns described.

Computational, functional, or logical components described and illustrated herein can be implemented in various forms, for example as hardware circuits, such as but not limited to custom VLSI circuits or gate arrays or programmable hardware devices such as but not limited to FPGAs, or as software program code stored on at least one tangible or intangible computer readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit Technology), or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device, and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or, more generally, by a suitable microprocessor, configured in accordance with methods shown and described herein, or any suitable subset, in any suitable order, of the operations included in such methods, or in accordance with methods known in the art

Any logical functionality described herein may be implemented as a real time application, if, and as appropriate, and which may employ any suitable architectural option, such as but not limited to FPGA, ASIC or DSP, or any suitable combination thereof.

Any hardware component mentioned herein may in fact include either one or more hardware devices e.g. chips, which may be co-located, or remote from one another.

Any method described herein is intended to include within the scope of the embodiments of the present invention also any software or computer program performing all or any subset of the method’s operations, including a mobile application, platform or operating system e.g. as stored in a medium, as well as combining the computer program with a hardware device to perform all or any subset of the operations of the method.

Data can be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary or off-line; which is of any type or level or amount or category of volatility, differentiation, mutability, accessibility, addressability, capacity, performance and energy use; and which is based on any suitable technologies such as semiconductor, magnetic, optical, paper, and others.

### DETAILED DESCRIPTION OF CERTAIN EMBODIMENTS

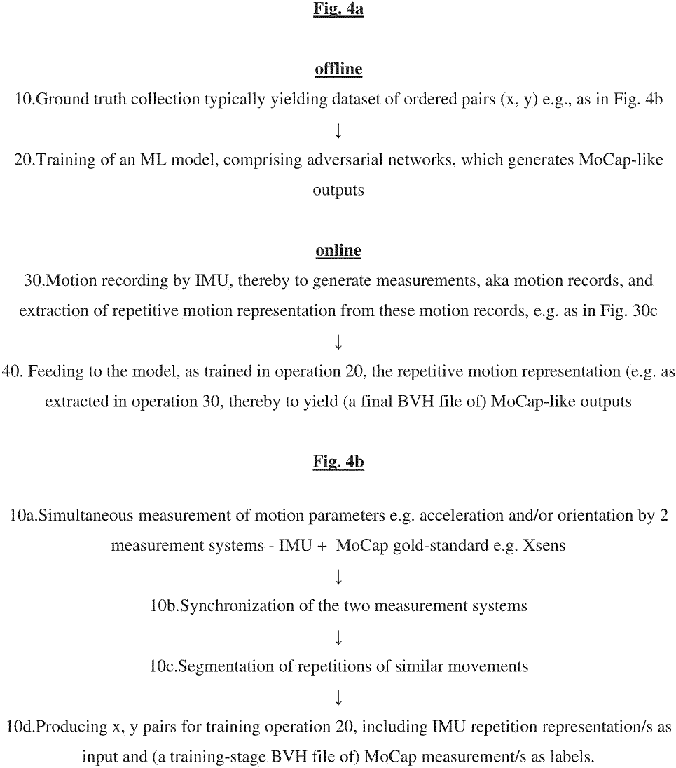

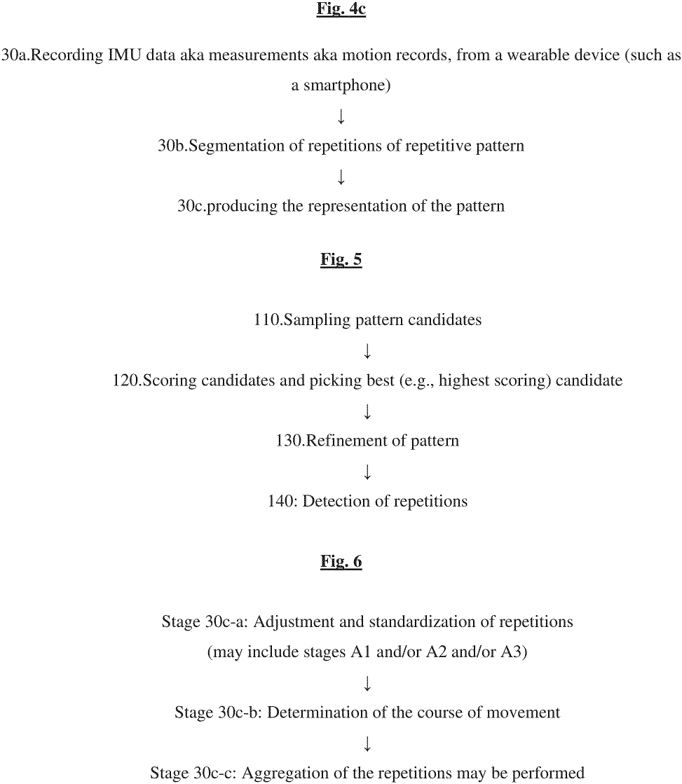

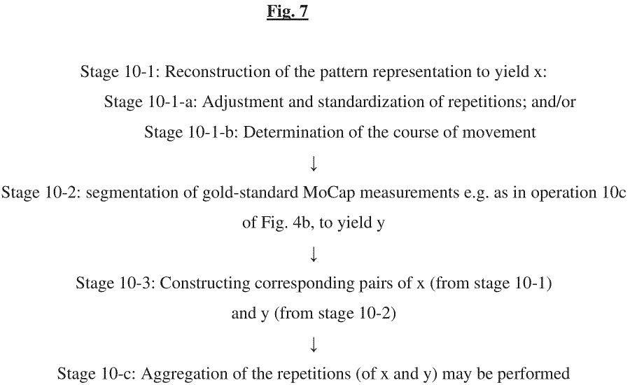

FIG. 4a is a simplified flowchart illustration of a method for generating estimated motion capture data for (at least) one first body portion performing a repetitive motion, using an IMU/s (Inertial Measurement Unit) deployed at a second body portion/s. Typically, ground truth is collected in offline operation 10; operation 10 may include all or any subset of operations 10a -10d, in any suitable order eg. as illustrated in FIG. 4b. This ground truth is used to train a machine learning model comprising adversarial networks in operation 20. Then, online, in operation 30, the IMU at the second body portion is used to record the repetitive motion, a representation of a single repetition is generated, and the trained model is used in operation 40 to generate the estimated motion capture data for (at least) the first body portion performing the repetitive motion. Operation 30 may include all or any subset of operations 30a – 30c, in any suitable order e.g. as illustrated in FIG. 4c.

Machine Learning may comprise training or teaching neural networks or models or algorithms to compute y as function of x e.g., to determine the coefficients of the regression function f, given that y = f(x) and x = IMU data. The label or tag or annotation may be given, or known from the ground truth, at the offline stage, vs. the outputs y (e.g., of the adversarial networks), e.g., at the online stage.

Ground truth typically includes x, y, pairs including x data comprising a representation of a body portion’s motion during a single repetition, and y data comprising sensor data generated by an IMU deployed on the body portion, during the single repetition, and wherein the x data is synchronized to the y data, such that the portion of the representation which describes the body portion’s motion at time t is paired to the sensor data generated by the IMU deployed on the body portion at time t.

Typically, each “pair” includes all data from sec 7 to sec 8:30, standardized, if 1 repetition takes 1.5 sec.

For example, if a subject walked 10 strides during MoCap, this will typically yield ten x, y pairs or samples for the ground truth database.

It is appreciated that non-repetitive motion may be regarded as a single repetition of a repetitive motion.

Offline Ground truth collection operation 10 may include an operation 10a in which motion by a human is recorded simultaneously by an IMU deployed at, say, the second body portion, and by a motion capture system which may also include a sensor deployed at the second body portion. The two resulting recorded tracks are typically synchronized to one another in operation 10b. Each track is segmented into single repetitions of the repetitive motion in operation 10c. Training set (x, y) pairs, including a representation of an IMU recording of a single repetition of the repetitive motion as x, and a motion capture recording of a single repetition of the repetitive motion as y, are generated in operation 10d.

Online operation 30 may include operation 30a in which IMU data is recorded using an IMU at, say, the second body portion, followed by a two-stage repetitive motion representation generation process including segmentation, followed by standardization. The two stages typically include segmentation operation 30b in which the resulting recording is segmented into single repetitions of the repetitive motion and standardization operation 30c which, responsively, provides standard representations to be fed into the trained model.

The adversarial networks typically comprise a first “generative” adversarial network which determines which candidate limb portion trajectories best fit the IMU measurements; and a second or “discriminative” adversarial network, competing with the first, which determines which candidate limb portion trajectories are physically feasible.

Physically feasible motion is a motion that is physically possible for a human to perform, or realistic, e.g., because a human’s body parts are physically capable of this motion, or are likely to perform this motion.

The recording is typically segmented into a motion capture output including a trajectory, for at least one body portion e.g., joint, which describes the individual body portion’s motion during a single repetition (aka cycle) of a repetitive motion that the human wearing the IMU is performing, using (inter alia) that body portion.

According to certain embodiments, the generative adversarial networks are used to derive a set of such trajectories, from the IMU measurements.

The first network may be trained to determine physical feasibility of at least one candidate body portion trajectory from among many candidate body portion trajectories. The second network may be trained to determine how well at least one candidate body portion trajectory, from among many, fits the IMU measurements.

It is appreciated that non-feasible trajectories, which are not physically feasible, may for example include all or any subset of the following:
upper limb portion (thigh) goes left and lower limb portion (lower leg/shin) goes right,
walking upside down
walking perpendicular to the floor
random motion in which a body part appears to flit erratically between locations –rather than smoothly or continuously transitioning from one location to another
walking upward into the air, as opposed to walking up a known hill
walking on water
movement of a joint that is not physically possible given the type of joint and/or known range of the joint’s motion e.g. person’s elbow bends backward/outward rather than folding inward, or a person’s calf appears to exceed the range of her or his extended knee; it is appreciated that some people who suffer from hyperextension of the knee, perform motion with just over 180 degrees of knee extension, but even such persons only slightly exceed 180 degrees. For example, elbow and knee joints only allow bending and straightening and some motions, e.g., rotational, which would be feasible for shoulder and hip joints which are ball and socket joints, are thus not feasible for hinge joints such as elbows or knees.

In contrast, any actual motion that was actually recorded, is considered feasible.

Typically, the training data used to train the adversarial networks in the model include only feasible motion (only motion that was actually recorded by an IMU worn by a human in motion).

The output of the generative model, aka generative neural network, typically comprises c=100 (say) X # of channels, assuming a cycle is divided into c units of time.

Eventually, each single c X r1 input representing a complete cycle or repetition is mapped to a c X r2 output, also representing the same repetition or cycle, where c is the number of channels in the input (each IMU sensor provides 6 channels), and m is the number of channels in the output. One embodiment of this method is described below, by way of example, not necessarily following the same notation.

Typically, the generative part of the model produces an output (motion capture data estimate) that both fits the IMU input and is considered feasible, out of input IMU data which may be random.

If a generative model is used, eg. stand-alone, using a more naïve approach, or as the generative part of a GAN, then typically, y, the output of the generative model, comprises trajectories (“MoCap data”) of 1 repetition and x comprises IMU data. It is appreciated that trajectories may be provided either for a single body portion or for a set of more than one body portions.

If a discriminative model (of the GAN) is used, then y, the output of the discriminative model, may have possible values within a range [0, 1] depending on the extent to which a given motion is feasible e.g. y is [0 — fake or non-feasible, 1 — real or feasible] ; and x comprises body portion trajectories (“MoCap data”) of 1 repetition.

It is appreciated that trajectories may be generated for any desired set of body portions such as but not limited to all or any subset of the following limb portions: left/right limbs, upper/lower (before/after joint e.g., before/after elbow/knee) limbs, arm/leg (total 8 limb portions), hand, foot, neck, torso/spine.

Example: the generative adversarial networks are used to derive a set of 4 trajectories, for a human’s right knee and ankle and left knee and ankle, from IMU measurements generated by an IMU in the human’s right hip pocket.

Regarding the x component of the pairs generated in operation 10d, typically, each representation of a single repetition of the repetitive motion performed by the person wearing the IMU includes IMU readings (acceleration and/or orientation change in each of 3 channels e.g.), taken over the duration of the single repetition, for each of 3 (typically) axes of motion. The axes of motion define a frame of reference, typically, forward motion is the positive x axis, and motion upward is the positive z axis. Typically the duration of the repetition is subdivided into (typically equal) time intervals or “units” such as, say, n = 100 such time units, and the x component (e.g. as output by operation 10d) includes an n x r matrix of the r IMU readings (typically each comprising a scalar) taken during each of the n time-units. For example, if the moving person is wearing a single IMU (in his right hip pocket, say), and there are 6 IMU channels, the x component may comprise a single matrix whose dimensions are 100 x 6.

Regarding the y component of the pairs generated in operation 10d, typically, each representation of a single repetition of the repetitive motion performed by the person wearing a MoCap uniform and IMU includes MoCap readings (acceleration in each of 3 channels e.g.), taken over the duration of the single repetition, for each of 3 (typically) axes of motion. The axes of motion define a frame of reference which is typically the same frame of reference as defined for the x component. Typically, the duration of the repetition is subdivided into (typically equal) time intervals or “units”, typically of the same lengths as for the x component, such as, say, n = 100 such time units and the y component (standard format of MoCap data representing a single repetition) includes, for each of several joints each of which bears a MoCap sensor, an n x r matrix of the r′ MoCap readings (typically each comprising a scalar) taken during each of the n time-units. For example, if the MoCap uniform includes MoCap sensors for each of 8 body joints and there are 3 MoCap channels, the y component of the pair may include 8 matrixes, and each matrix’s dimensions may be 100 x 6, which form a conjoint matrix that has a dimension of 100x48.

All or any subset of the following three categories (or any relevant subset of each) may be provided as an output):
F1. The hip joint (of the leg on which the phone is), 3 channels of flexion, abduction, and rotation.
F2. Lower body kinematics, including lower back, l/r hip, l/r knee, l/r ankle, l/r toes X 3 channels each (flexion, abduction, and rotation) = 27 channels of kinematics.
F3. Full body kinematics: upper body kinematics typically including 4 spinal joints, neck, head, l/r shoulders, l/r arms (3 joints each) include 14 joints, plus the lower body kinematics, total = 69 channels of kinematics.

Whether to provide F1, F2, F3, or all, or a combination thereof, typically depends on the use case, and may therefore be made as an engineering decision for the general settings of the system, or may be provided as a configurable option. For example, F1 may be useful for estimating the hip joint range of motion for clinical assessment of the hip joint. F2 may be useful for simulating the entire lower body movement, since graphs and numbers provided by F1 may be hard for users to interpret, and use of F2 may provide both broader and easier perspective of the subject’s movement. F3, the most demanding option in terms of ML feasibility, provides complete analysis of the movement. The F3 settings may require the subject to wear a full body uniform, which may be inconvenient.

The term kinematics as used herein is intended to include any trajectory of any body part, such as but not limited to joint trajectories. Kinematics may or may not be represented in BVH format.

Motion capture (MoCap) usually refers to motion labs recording the movement of objects or people, used in medical, entertainment, sport, and other applications. This description explains how to extract (human or other subjects) locomotion representation from a gait cycle measured by an inertial measurement unit (IMU) placed in a single bodily position. Rather than walking, a gait cycle refers to any repetitive motion activity a subject can perform (such as stairs climbing, running, jumping, etc.).

Offline extraction of (human or other subjects) locomotion representation from a gait cycle may employ a machine learning (ML) scheme, and may include:
Collecting ground truth data for training and validation - typically including at least two operations: operation 10a which may provide simultaneous measurement of the IMU recording, and external MoCap gold standard measurement systems, and operation 10b which may include andSynchronization of the two measurement systems to provide pairs each including an IMU repetition representation as input and a MoCap measurement as a label.
And/or
operation 20 - Training of an ML model, comprising adversarial networks, which generates MoCap-like outputs.

A MoCap’s standard representation of locomotion, which may serve as the y component of the pairs produced in operation 10d and may be used to train the model in operation 20, is now described in detail. It is appreciated that each BVH file may, for example, store one MoCap’s standard representation of locomotion. BVH is of course only one format from among many which are known for storing motion capture data Typically, in operation 10d, the pairs each include an IMU repetition representation as input and a MoCap measurement as a label. The former may include a standard representation of a single repetition as measured by the IMU, and the latter may include a standard representation of a single repetition as measured by motion capture.

The use and importance of gesture and motion data (e.g. IMU data or MoCap data) are useful in haptic systems, controllers and motion capture systems, etc. with the development of virtual reality, gaming, film, and medical appliances. These signals, including all sensor data and information about the sensors and the user, are stored in many different formats, one of them being the BioVision Hierarchy, or BVH, developed by Biovision for motion capture.

The BVH structure, e.g, as shown in the Appendix A below, typically includes:
a. Header - may include a skeleton hierarchy, including a description or declaration of all joints and/or their hierarchical connections, and/or their relative offsets and/or the channels each is using; and/or the length and rate of data to follow.
b. Data - the stream of motion data as channels per joint in the skeleton, typically comprising the kinematics, which quantifies the different angles of the joints, and the spatial position of the joints. Usually starts with T-pose before the actual recorded data.

As can be seen in Appendix A - an example BVH file, the hierarchical skeleton has its origin set at the hips, with 4 spinal reference points between the hip and the neck plus one for the head, and 4 points for each limb at each joint - collar, shoulder, elbow and wrist for the arms; and hip, knee, ankle and toe for the legs. Each of these joints registers 3 channels for the Euler angles at YXZ convention, while the origin at the hips also registers 3 channels for its position in a right-hand coordination system - X-left, Y-up, Z-forward.

This skeleton may be used for all various motion BVH outputs e.g. if it is assumed that every subject has the same skeleton, such that the only difference between subjects’ measurements has to do with motion, and so creating BVH files (e.g. in operation 40) typically only requires extracting motion data e.g., IMU data or MoCap data or kinematics for each of plural body portions e.g. joints.

A method for detection and segmentation of a repetitive pattern (e.g., in the course of performing operations 10c and/or 30b) is now described in detail.

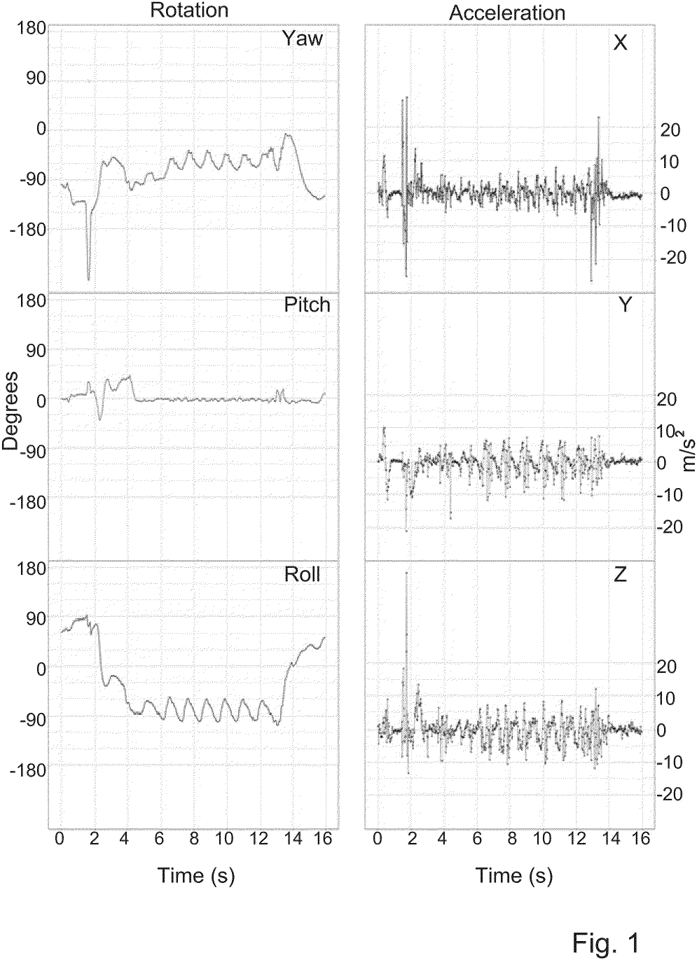

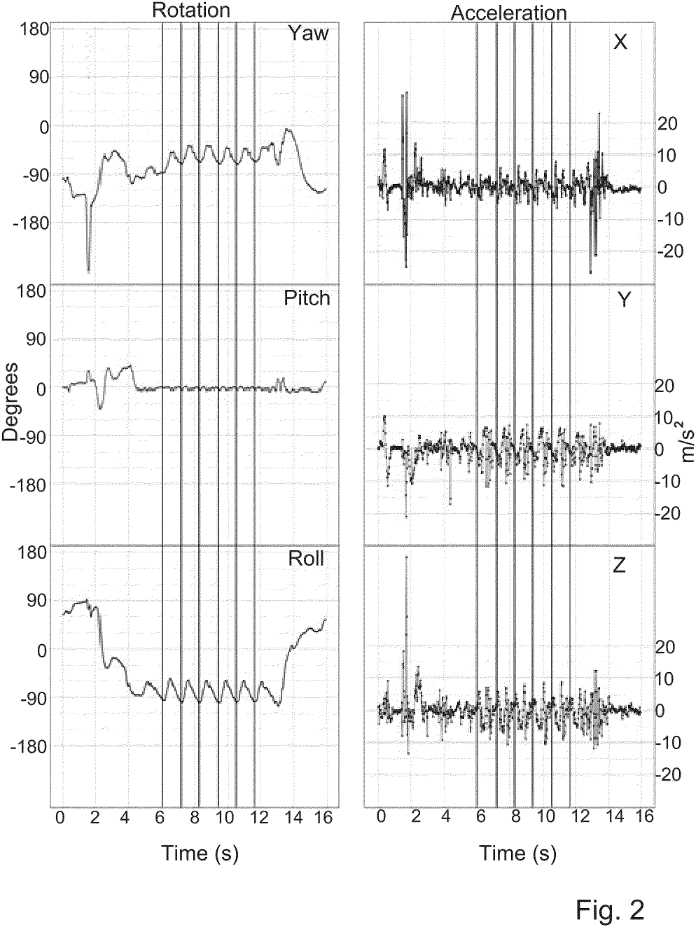

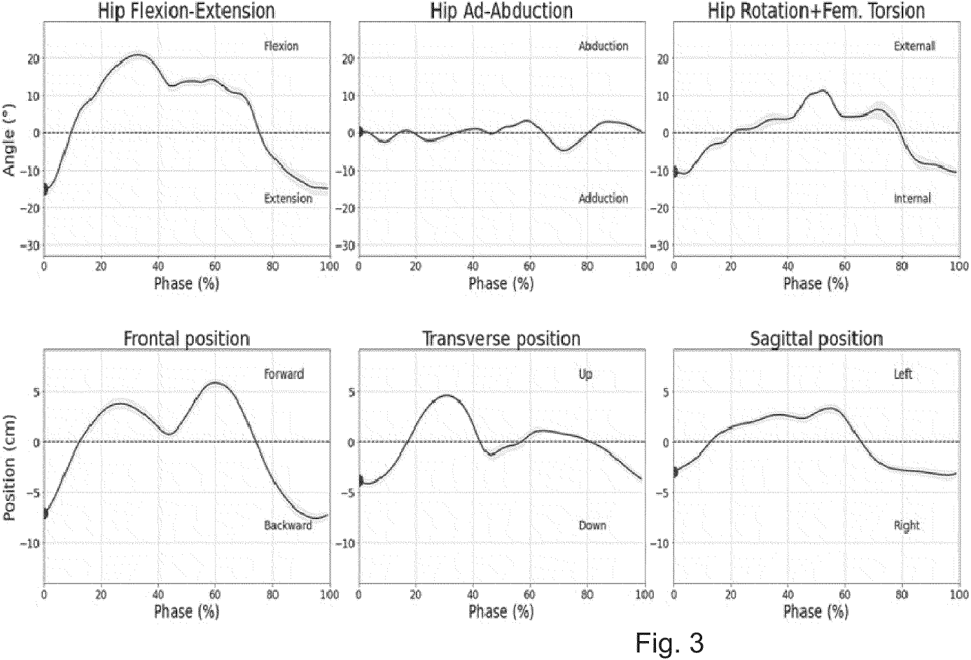

Any suitable conventional method for segmentation of a repetitive pattern may be employed, and FIG. 2 herein, taken from co-pending USSN 17/500,744 filed Oct. 13, 2021, the disclosure of which is hereby incorporated by reference, presents the results of segmentation of a repetitive pattern over the same motion record shown in FIG. 1 herein, also taken from the same co-pending USSN 17/500,744.

FIG. 5 is a simplified flowchart illustration of an example method which may for example be used to perform operations 10c of FIG. 4b and/or 30b of FIG. 4c. The method first identifies a repetitive pattern. The method typically comprises all or any subset of the following stages 110-140:

Stage 110: Sampling pattern candidates, each of which comprises a data segment. It is appreciated that it may not be known a priori, which candidate data segments actually exhibit the repetitive pattern, hence are “successful candidates”. For example, it may be desired to identify a gait cycle which is the repetitive pattern which a patient is walking. However, some data segments (IMU recordings over an interval of time e.g.) may be “unsuccessful candidates” because they turn out to have been recorded when the patient was sitting or standing, rather than walking. Also, the length of a gait cycle (pattern duration), for various subjects, may vary from 0.5 seconds to, say, 4 seconds. Thus, a segment of a given length such as 2 seconds, may represent one subject’s entire gait cycle, half of a cycle for another subject, and 2 or 3 cycles for a third, fast moving patient.

Typically, the repetitive pattern is a segment (eg., sequence) of the original (eg., IMU) data which occurs several times. Thus, finding the pattern by sampling is doable. Sampling of candidates may comprise taking data segments with a fixed duration every x seconds. In order to reflect a significant pattern, the candidates may have a long enough duration. For example, a 2-second duration may be used, sampling candidates every 4 seconds.

Stage 120: Scoring the candidates with a score which reflects the objective of this process which is finding a pattern that describes the data well e.g., a pattern which, if repeated, at the indexes of repetitions, this reconstructs the original data accurately e.g., with minimal or below threshold Euclidean distance between the original data and the reconstructed data. Therefore, the better and more accurately the candidate covers the data, the more preferable that candidate is. For instance, the correlation of the candidate with the data may be computed over time over each of the channels. The total correlation over time is the mean correlation of all of the channels’ correlations. Peaks of the total correlation over time are detected with conventional signal processing methods such as the peak-finding methods described here: (https://docs.scipy.org/doc/scipy/reference/generated/scipy.signal.find_peaks.html), configured to filter in only peaks with the highest values over a sliding window of fixed duration. Peaks with high correlation values imply a repetition of the pattern, however the repetition may be shorter than the duration of the pattern, since correlated data intervals may overlap with one another. Hence, the real repetition is either the data interval that matches the pattern duration, or the interval between correlation matches (peaks with high values of correlation), typically the shorter of the two. Eventually, the score may be computed as a sum of the repetition durations multiplied by their correlation values which may be regarded as reflecting the measure of coverage that the pattern candidate captures. A candidate is dismissed if no sequential repetitions are detected, indicating that this pattern is not repetitive. Parameters configured for this process may include peaks’ sliding window duration parameter which may be determined by the shortest repetition expected; e.g. the parameter may be 0.4 seconds, as capturing a significant pattern with higher frequency than this value enables, is not expected. Other parameters configured for this process may include the correlation threshold to consider a peak as a potential repetition, and/or the minimal number of consecutive repetitions, and/or the maximum deviation of the intervals’ durations between consecutive repetitions. Example parameters’ values may be correlation threshold: 0.5, minimal number of consecutive repetitions: 3, maximum deviation of the intervals’ durations between consecutive repetitions: 40% of deviation from the average duration.

Once the candidate with the highest score has been identified, all or any subset of the following stages may be carried out, suitably ordered e.g as follows, typically , in operations 10c and/or 30b:

Stage 130: Refinement of the pattern - the segments between consecutive repetitions have the correct length of the repetitive pattern; moreover, their average pattern may be shown to achieve a higher average correlation with all the repetitions. So the average of the segments between repetitions would get a higher score as a candidate, and therefore may be deemed the “true” repetitive pattern. All segments may be cut to fit the shortest interval between repetitions, to avoid overlaps.

Stage 140: Detection of the repetitions - find peaks of the total correlation between the final pattern and the original data, e.g. as was done when evaluating the scores of the pattern candidates; the same configured parameters as before may be used again. Only intervals between consecutive peaks as repetitions may be considered; although this might miss the first and the last repetitions of a sequence or sequences of two repetitions, this ensures that the considered intervals are real instances (repetitions of) the repetitive pattern. In addition, the first and the last repetitions in a sequence might be slightly different than the rest of the repetitions. Thus, typically, precise detection of real repetitions is prioritized over “covering” all repetitions (over the goal of detecting every single repetition).

A method for generating a repetitive pattern representing an IMU’s (or motion capture system’s) recording of a single repetition of a repetitive motion, e.g., by segmentation, is now described in detail. All or any subset of the operations included in this method may be used e.g., by operations 10c and/or 10d and/or 30b and/or 30c.

FIG. 7 is a simplified flowchart illustration of a method for reconstruction of the pattern representation which may be used standalone or to implement operation 10d in FIG. 4b.

The method of FIG. 7 typically includes all or any subset of the following stages 10-1, 10-2, 10-3 and 10-c (the following description may be augmented or replaced by the disclosure of co-pending USSN 17/500,744 filed Oct. 13, 2021, incorporated herein by reference):
Stage 10D-a (aka stage 10-1-a)-Adjustment and/or standardization of repetitions typically includes the following operations A1 - A3, which may be performed for each repetition:
A1. Ignoring the offset of the yaw channel, embed rotations in uncalibrated reference frame
A2. Displacement and orientation adjustment:
   Computing average acceleration over the repetition, subtracting from the accelerations, and integrating the accelerations over the segment to get velocities;
   then subtracting the average of those velocities from each of those velocities, and then integrating the velocities over the segment to yield displacement.
   Orientations may be handled after the reference frame has been fixed:
A3. interpolation - interpolate the displacement and the orientation of each segment to have (say) 100 equal time units, to ensure a standard structure (same number of time units) for each repetition

Stage 10D-b (aka stage 10-1-b)- Determination of the course of movement

Stage 10D-c (aka stage 10-c) - Aggregation of the repetitions may be performed e.g., by computing a central measure, such as an average, for the repetitions, for both the IMU data and for the MoCap data. Thus, the output of this stage, may comprise an average of plural individual repetitions (of several from among the 100X6 samples e.g.) which may be provided instead of individual pairs of repetitions. According to certain embodiments, instead of providing individual repetitions (either x or y), an average of plural corresponding repetitions (for x and y) may be provided, which reduces some noise, possibly at the expense of shrinking the dataset and/or eliminating or downplaying rare samples.

Typically, an ML model is generated, that inputs an IMU gait representation and outputs a MoCap standard locomotion representation which may use generative adversarial networks.

The output of operation 10d typically comprises a 100 (say) x C matrix, assuming division of the repetition into 100 time units and C (e.g. 6) channels.

### Bodily Positions of the IMU Sensor

The following operations may be performed plural times, with the IMU device deployed in plural bodily positions respectively. For each position, another ground truth dataset may be gathered in operation 10, and another ML model may be trained against that dataset in operation 20, typically using the same structure of model but a different training set, yielding a different model for the corresponding bodily position (e.g., in operations 20 and/or 40).

It is appreciated that extraction of MoCap-like outputs may include segmentation of ground truth data and/or of IMU data collected online (e.g., in operations 10c and 30b respectively) into repetitive patterns, each of which typically comprises a stride, by one of, or both of, of the patient’s two feet.

One known method for segmentation of a motion record, into strides which may be used to perform operation/s 10c and/or 30b, is described in co-pending published U.S. Pat. Application re Assessment of a User’s Gait (Application #20200289027 issued Sep. 17, 2020), whose disclosure is incorporated herein by reference, which describes segmentation of a motion record, into strides. Specifically, this co-pending patent document describes “Operation c-d: Segmentation into Strides” as follows:
“Many methods for stride detection are known; acceleration data is typically segmented along the time axis, of into strides. Methods for performing such segmentation are described e.g., in:
Perez, Andres A., and Miguel A. Labrador. “A smart phone-based system for clinical gait assessment.” 2016 IEEE International Conference on Smart Computing (SMARTCOMP). IEEE, 2016; and/or
Gadaleta, Matteo, and Michele Rossi. “Idnet: Smartphone-based gait recognition with convolutional neural networks.” Pattern Recognition 74 (2018): 25-37. Alternatively, rotation data may be segmented, along the time-axis, into strides. Any stride detection technique may be employed. Described herein is an example of a heuristic method to extract segments of strides from rotation data; alternatively however, the methods of Perez/Labrador or Gadaleta/Rossi may be employed.
An example of stride segmentation results generated from the motion record rotation data in FIG. 2b of Published U.S. Application #20200289027, is shown in FIGS. 3a-3b of Application #20200289027.
Example Pseudo-code for segmentation into strides; all or any subset of lines of the following code may be used:
Let Acc ∈ ^{3Xn} be the acceleration data Let Rot ∈ ^{3Xn} be the rotation data //Check if the motion record is not static 1. velocity = cumsum(Acc, axis=1) 2. energy = sum(sum(velocity^{∗∗}2, axis = 0, axis = 1) 3. if(energy < energy_ threshold): 3.1. return None //Ignore the yaw component of Rot 4. smooth __ Rot = Rot[1:,:] //Make rotation smoother with convolution, mask size is 0.25 second in sampling rate unit 5. smooth_Rot = conv(smooth_Rot, Gaussian1D(0.25 ^{∗} freq)) //Take the main component of rotation deviation 6. smooth_Rot = PCA.fit_transform(smooth_Rot)[0,:] Finding extremum point is straight forward 7. extremum indexes = find extremum points(smooth Rot) 8. extremum indexes a = extremum_indexes[::2] 9. extremum_indexes_b = extremum_indexes[1::2] 10. range_a = std(smooth_Rot[extremum_indexes_a] 11. range b = std(smooth_Rot[extremum_indexes_b]) 12. if(range_a >range_threshold OR range_b >range_threshold) 12.1. return None 13. duration_a = std(diff(extremum_indexes_a)) 14_{.} duration_b = std(diff(extremum_indexes_b)) 15. if(duration_a >duration_threshold OR duration_b >duration_threshold) 15.1. return None 16. strong_forces =sum(Acc^{∗∗}2, axis=0) //Sum forces locally (0.25 second) 17. strong_forces = conv(strong_forces , Rect(0.25 ^{∗} freq)) 18. sum_force_a sum(strong_forces[extrernum_indexes_a] 19_{.} sum_force_b = sum(strong_forces[extremum_indexes_b] 20. if (sum_force_a > sum_force_b) 20.1. return extremum_indexes_a 21. return extremum_indexes_b″
it is appreciated that the (typically BVH-formatted) output of the MoCap system is divided t into repetitions (e.g., using the time intervals from operation 10c) to provide the y component.

An example method for identification and representation of a repetitive pattern_measured by IMU is now described with reference to FIGS. 1-3 herein, taken from co-pending USSN 17/500,744 filed Oct. 13, 2021, the disclosure of which is hereby incorporated by reference. All or any subset of the operations of the method may be performed in the course of operation/s 10c and/or 10d and/or 30b and/or 30c. Specifically, the method is typically configured to identify a repetitive pattern in sensor data measured by an inertial measurement unit aka “IMU data” or to generate a standard representation of that repetitive pattern, if a repetitive pattern exists, and to utilize properties of the standard form for analysis. A repetitive pattern typically implies that while the IMU data was being generated, the IMU underwent specific repetitive motion activity e.g., because the sensor was worn by an end-user who was, as the data was being generated, engaging in walking, running, exercising, or any other movement composed of repetitions. Typically, the IMU data undergoes standardization e.g., as described herein, typically before the repetitive pattern, if any, is detected and, if detected, segmented. Reconstruction of the standard representation of the pattern (e.g., as performed in operations 10d and/or 30c), if any, typically includes: adjustment and/or standardization of IMU data on repetitions, and/or determination of the course of movement, and/or aggregation of the repetitions.

FIG. 3 for example graphs a 6-channel representation of a repetitive motion pattern. The 3 upper graphs show the 3 angular channels, their names having been inspired by the example of a walking man, while the motion is measured from the pants front pocket. Since the motion reflects the movement of the thigh, the first angle graph approximately represents the flexion and the extension of the hip joint, the second corresponds to the abduction movement, and the third one approximately shows the total rotation of the thigh and the body. The 3 lower graphs show 3 displacement channels of which the first corresponds to the movement forward, the second is the vertical displacement, and the third is the horizontal displacement.

It is appreciated that the IMU may use a combination of several sensors, including all or any subset of accelerometers, gyroscopes, and sometimes magnetometers and other sensors such as a barometer, to provide, typically, at least three channels of spatial orientation and three channels of acceleration over time in various sampling rates with, typically, at least a frequency of 50 Hz or higher. The IMU utilizes some of its measurements to eliminate gravitational forces from the IMUs′ output, and different algorithms (such as a Kalman filter) to produce an “elementary data stream” comprising more accurate channels of orientation and acceleration (e.g., without the gravitational influence) at 50 Hz at least. Additional channels such as altitude overtime may be provided, and, if so, is considered extra information. An example of a data record of a 16 second time window is shown in FIG. 1 herein, taken from co-pending USSN 17/500,744 filed Oct. 13, 2021.

Raw data may be standardized in terms of continuity of measurements and/or in terms of consistency of interval between measurements. The IMU device orientation may be measured by the IMU device’s rotation in yaw, pitch, and roll, from a given direction e.g., north. Most sensors produce rotation angles in bounded range (for example, the yaw angle is between -180 to 180 degrees), so when the rotation exceeds the range, it starts over. This operation may be reversed to yield data which is continuous, as is reality. This may be seen in the upper left portion of FIG. 1 herein, taken from co-pending USSN 17/500,744 filed Oct. 13, 2021; as the yaw angle drops down and exceeds the value range, the difference between the “fixed” raw data in dots, and the standardized reversed data as a continuous line, is apparent. Any suitable method may be employed to standardize (reverse the “starting over” operation), such as, for example: each time a discontinuity is detected, using some suitable definition of discontinuity such as a difference between two adjacent data points which exceeds a threshold such as, say, 180 degrees, the system may add or subtract 360 degrees to eliminate this difference, or to bring the difference between the threshold.

Motion sensors ostensibly have determined sampling rates, however it turns out that in practice, their sampling points of time are not exact. Converting the raw data into consistent intervals, i.e., a sequence of values in fixed points of time spaced equally in a specific rate, may be accomplished by interpolation of the data in time. Fortunately, a sensors’ possible sampling rate is about 100 samples per second, and may thus be converted to, say, 50 samples per minute. Linear interpolation is typically good enough, e.g., as may be seen in FIG. 1, taken from co-pending USSN 17/500,744 filed Oct. 13, 2021, the raw data fit the standardized data very well.

A method for reconstruction of the pattern representation, which may be used, for example, by operation 30c in FIG. 4c, is now described with reference to FIG. 6. The method typically comprises all or any subset of stages a - c (aka stages 30c-a, 30c-b, 30c-c) which are now described.

The measurement may be represented in a reference frame defined as follows: the X-axis is the course of the movement of the IMU (of the end-user wearing the IMU e.g.), the Y-axis is, say, toward the sky, the Z-axis is, say, toward the right (the normal of the X-Y plane). The following stages a - c yield a representation of the repetitions which typically capture all relevant information of the motion being measured while being invariant to irrelevant attributes of the measurement and the analysis, including the base orientation at which the IMU device was set during the measurement, and phase 0 at which the repetitions define the starting point of the repetitive pattern. The output of the following 3 stages typically comprises a standard representation of the pattern, including 3 spatial channels, 3 orientational channels, and a channel for each extra information channel of the original data, with over 100 uniform units dividing the duration of the repetitions.

### Stage a (Aka Stage 30c-a)-Adjustment and Standardization of Repetitions

For each of the data segments of the repetitions, perform all or any subset of the following operations, suitably ordered e.g., as shown:
a-1: Ignoring the offset of the yaw channel - the north is irrelevant to defining the frame of reference as defined above, and similarly any other offset of the yaw, since given a motion record of someone walking, the direction of the walk is not relevant, and it is desirable for the representation of that activity to be invariant of the direction. Hence, the average yaw may be subtracted for each repetition, whether the data is calibrated to the north, or showing a false reference of the north. Other techniques may also be used, such as splitting the yaw channel into two - one channel for the azimuth (a smoothed version of the yaw represents the macro changes of the yaw), and the other channel for subtraction of the azimuth from the yaw, which represents the micro changes of the yaw.
a-2. Embed data in uncalibrated reference frame e.g., as described elsewhere herein. Rotating (using conventional rotation arithmetic) the acceleration channel with the corresponding orientation from the orientation channel produces acceleration in the global reference frame of earth (X=north, Y=west, Z=towards the sky). Multiplying the orientation channel with their inverse mean of orientation on the right produces rotations that are relative to the mean orientation of the segment, aka the relative orientations. By rotating the acceleration and producing the relative orientations, the relation between the data and the orientation of the measurement system (the IMU device) over the body is eliminated, and what remains is accelerations and relative rotations in the uncalibrated reference frame, e.g., a reference frame in which the X is, say, the north rather than the course of movement. Since the yaw channel is neutralized in operation a-1, X is an arbitrary north rather than the geographical north. Typically, neither the geographical north nor the arbitrary north are helpful for defining the course of movement, hence the method may neutralize the north to render the remainder of the process independent of the course of motion in the global reference frame.
a-3. Displacement and orientation adjustment - typically, the data is presented as displacement, although the data could also be presented as accelerations. The displacement is smoother, enables understanding of the motion better for human eyes, and preserves the information of the original data, which may be reversed by derivating or computing a derivative of the displacement. The reference frame moves with the movement, meaning that the displacement over the data segment may be assumed to be 0 which determines the initial velocity (the velocity may also be assumed not to change over the data segment, meaning that there are no accelerations between repetitions, which turns out to be a reasonable assumption for long continuous repetitions. In addition, the effect on the data is minor, which is subtraction of the average acceleration over the data segment (the average acceleration may be kept). This assumption yields benefits: the data may be fixed due to noise or biases of the sensors which are reasonable, and the displacement is not only closed (as it starts and ends at the same displacement) but is also smooth, since the velocity is the same at the beginning and at the end. This makes any shift of data from the end of the segment to its beginning yield the same displacement, yielding invariance to the determination of phase 0, the starting point of the repetitions’ segments. The orientation of the starting point and the last point may be constrained to be the same.

Thus, according to certain embodiments, the average acceleration is subtracted from the accelerations and integrated over the segment to get velocities; then the average velocity is subtracted from the velocities and integrated over the segment to get the displacement. Orientations are handled after the reference frame has been fixed.

a-4. Interpolation - interpolate the displacement and the orientation of each segment to have (say) 100 equal time units, to ensure a standard structure for each repetition (with an equal number of time units).

### Stage b (Aka Stage 30c-b) - Determination of the Course of Movement

Rotate the reference frame such that X and Z axes will be calibrated, where the course of movement is the X-axis. This rotation depends on the orientation of the measurement system referred to the motion itself; typically, all of the repetitions’ representations at this stage are embedded in coherent reference frames, which may need to be calibrated with the same rotation. This rotation may be estimated using any suitable technique, such as applying principal component analysis (PCA) on the projections of either the displacements or the orientations on the horizontal plane (which is known, since the vertical axis is known). The most significant component of the displacement may imply the direction of the movement because this is the direction in which the most significant changes typically occur. Similarly, the most significant component of the orientations represented as rotation vectors may imply the Z-axis around which the most significant changes of orientations typically occur (e.g, in walking measured from the thigh (e.g., if the IMU is adjacent to the end-user’s thigh), the most significant component of the orientations over the horizontal plane is the hip flexion-extension axis). Either of these (the most significant component of the displacement, or the most significant changes of orientations) may be used to rotate the reference frame. Eventually, this yields all of the data represented in a calibrated frame of reference. The orientations may then be converted to Euler representation in the order of Z-X-Y, and the average angle velocities of each orientation channel may be subtracted to verify each of them starts and ends at the same angle, which yields invariance to the initial point of the segmentations e.g., as described above.

It is appreciated that the above process may be used to implement stage 10-1-b of FIG. 7, and may also be performed stand alone.

### Stage c (Aka Stage 30c-c) - Aggregation of the Repetitions

The repetitions’ representations are similar to each other, even initially, and, typically, after standardization of their structure and elimination of irrelevant attributes of measurement and analysis, the repetitions’ representations become even more similar. Some tasks may use one aggregated representation of the motion, rather than on each of the repetitions, for example, recognition of the activity being performed by the subject and the position of the measurement device e.g., IMU as well. For this kind of task, the aggregated form of the repetitions’ representations may be employed. This process may, for example, comprise naive averaging each of the channels over every one of 100 (say) time-units or by dynamic time warping (DTW) techniques. In practice, the naive aggregation method, while less sophisticated, nonetheless works well.

It is appreciated that, alternatively, the data segmentations (e.g., in operation 10d) may be taken as-is, and stages a, b, c may be omitted.

### Ground Truth Collection (Operation 10)

In this operation, a ground truth dataset is generated, for use in training e.g., as a training set, and/or for validation including determining accuracy of models. If dataset of n samples = (x,y)ⁿ, then, according to any embodiment, an IMU repetitive pattern representation / an IMU segment = x and/or an Xsens/BVH segment = y. It is appreciated that x may be plural IMU measurements or but one such measurement. Typically, the ground truth dataset includes (x,y)ⁿ pairs where each x is an IMU repetitive pattern representation or IMU segment and each y is an Xsens segment or a segment generated by any other gold-standard MoCap system, typically in BVH format.

The ground truth dataset typically comprises plural samples of repetitive motion representation (which may be generated by segmentation operation 30b) as input and their corresponding MoCap outputs (repetitive human locomotion representation which may be generated by segmentation operation 10c) as labels. Each label typically includes kinematics (e.g. in BVH format) corresponding to the time interval of the IMU segments. The ground truth is typically a representative sample of the reality and may challenge the ML models as much as the reality may challenge their accuracy. It is recommended that the ground truth will include hundreds of subjects, a good representation of the general population, performing various kinds of walks by different instructions (asymmetrical walks with different step lengths for the right and the left leg, imitations of various limitations such as walking on the toes in one leg, or on the heels only, or not bending the knee, etc.), known as the measurement protocol. Each portion of a specific walk in which a subject performed a given instruction may include plural e.g., at least 20 repetitions, before the subject is given a different instruction, and each subject’s movement may be measured from different bodily positions. In total, the ground truth sample may include (say) hundreds of thousands, or more, pairs of repetitive motion representations and labels, where both members of each pair are typically generated by segmentation.

### Gold Standard MoCap Measurement e.g., for Operation 10a

When generating a MoCap gold standard measurement, e.g., in operation 10a, the Xsens MoCap system may, by way of non-limiting example, be used; this is a wearable technology to measure a subject’s gait. Manufacturer’s instructions may be followed to install the uniform and the different system’s parts on the subjects, and perform a calibration to their body measurements. After that, the system is ready to record the subject’s motion, and hence, recording IMU data may commence using smartphones placed in various bodily positions (such as pants’ pockets). Subjects are instructed to perform different gait patterns (such as, say, all or any subset of walking with long/short steps, long horizontal distance between feet, different back postures, different postures of the feet while touching the ground, different speeds, different cadence, different flexion of the hip joint, no flexion of the knee), while the subject’s motion is recorded on the smartphones and the Xsens system. The system control software is marked when new instructions are provided and the gait pattern changes. This stores timestamps of changes of gait patterns over the recording. Eventually, MoCap recording and IMU recording of the same activity is obtained, with additional timestamp markers of change of gait.

### Measurement Synchronization e.g., for Operation 10b

In order to provide models, e.g., for training purposes, with pairs of IMU-repetitive motion representation, typically generated by segmentation, and their corresponding lower body kinematic data, the motion capture sensors and the IMU system are typically synchronized.

There are several ways to synchronize the systems, such as calibration of the gold-standard MoCap’s and IMU’s respective internal clocks with each other, or figuring out the offset between the gold-standard and IMU, if the reference system’s (e.g. gold standard system’s) internal clock is accessible. Another alternative, e.g. if the internal clock is not accessible, is a high-frequency (e.g. FPS (frames per second) higher than 100) video that captures the internal clock presented by the IMU measurement device (e.g. the IMU is installed in a smartphone, and the IMU’s measurement clock is presented on the smartphone’s screen), the events being measured by the reference system. By detecting the first temporal event measured by the reference system in the video, the system’s clock offset which refers to the video time may be found, and the offset of the video with the IMU device may be found by extracting the IMU device clock’s timestamp shown on the video.

According to another embodiment, the approach is to correlate the signals from both sources, as both systems typically provide acceleration and orientation channels for the lower body. This may comprise creating an acceleration amplitude signal from both sources of data taken from a single bodily position (say the hip), and calculating cross-correlation, adjusting the measurements so that the peak of the cross-correlation occurs, say, at time 0. Typically, the two systems operate at different rates, but using, say, interpolation to ‘fill the gaps’ of data yields sets of similar signals synced with high enough precision.

After syncing the signals in operation 10b, the data is segmented in operation 10c. In operation 10c, typically, the raw IMU data representing repetitive motion -is segmented into intervals, then the segmentation is applied to the kinematics signal, to create segments of motion capture data.

### Generating an ML Model (e.g., for Operation 20)

Methods for ML model generation, which may be used to implement operation 20 thereby to yield a trained model which may be used, for example, in operation 40, are now described.

According to one “naive” embodiment, an ML model that inputs an IMU gait representation (e.g., resulting from segmentation) and outputs a MoCap standard locomotion representation, which typically represents a single cycle of a repetitive motion, may use a conventional (rather than adversarial) neural network, using the general strategy of trying to minimize the difference between the generated and the target kinematics. In this approach, the ML may include many different layers and/or may include 1-dimensional cyclic convolutional layers that seek to mimic the relation between the input and the output, but as the performance is measured on being close to the output, this can yield an ML that gives very close results numerically, but that does not look “natural”, and/or, may overfit the ground truth.

According to another embodiment, a generative adversarial network is employed. Generating an ML model that inputs an IMU gait representation and outputs a MoCap standard locomotion representation (e.g., in operations 20 and/or 40) may use generative adversarial networks (GANs).

In this method, two models compete in the process of generating new motion capture data, and discriminating the generated data from the original MoCap data. Conventionally, in a GAN scheme, the generative model is trained on random input e.g., a random seed for the generative model. In contrast, the input (the x component) may include a repetitive motion representation produced from an IMU device rather than including random input, and thus generates data ‘out of thin air’ so to speak; this data looks genuine if trained correctly with the discriminator, yet represents no actual motion in real life. The discriminator is usually only exposed to the output of the generative model as false examples of input, and to real life examples as true input to learn.

More specifically, the following describes how, according to an embodiment, the adversarial nets’ models may be trained to produce MoCap-like outputs responsive to receipt of IMU data, in operation 20, including the structure of the models which are trained, according to certain embodiments.

The result of operation 20 typically comprises an ML algorithm that gets IMU data and produces predictions of MoCap outputs. Different approaches of ML models can be taken to implement a function that inputs an IMU gait cycle representation and outputs a MoCap standard locomotion representation (e.g. by segmenting motion capture data representing a repetitive motion). The following are descriptions of such two NN structures: the naive NN implementation, and the generative adversarial network (GAN) implementation.

Naive implementation: ML model may use a “simple” neural network, using the general strategy of trying to minimize the difference between the generated and the target MoCap data. In this approach the ML can be very elaborate, and may comprise various, e.g. many different, layers (in particular, 1-dimensional cyclic convolutional layers) that try to imitate the relation between the input and the output, but as the performance is measured on being close to the output, this can yield an ML that gives very close results numerically, but that does not look “natural”, or, furthermore, this can overfit with the ground truth.

Generative adversarial network implementation: ML algorithm includes two models which “compete” in the process of generating new motion capture data and discriminating the generated data from the original MoCap data. Typically, two models are trained simultaneously:
1. The discriminative model, which classifies whether an input is a realistic or feasible sample or not, is usually only exposed to the output of the generative model as false examples of input, and to real life examples as true input to learn.
2. The generative model, which produces samples, is typically trained to increase the error rate of the discriminative model. The generative model may be trained on random input, and generates data which looks genuine if trained correctly with the discriminator, but represents no actual motion in real life.

To train these two models, the discriminator may first be compiled with its own loss function. Then a new model — the GAN — is compiled as a sequence of the generator model followed by the discriminator model, where the discriminator is not trainable. In the training process, each epoch starts with the discriminator training on two sets - real outputs labeled as True, and synthetic outputs labeled as False. This teaches the discriminator to distinguish between actual outputs and what the generator model generates. Later, the whole GAN is trained using the model output e.g., the discriminator output, on some random seed input, as the loss for the GAN model. Typically, the discriminator is not trainable as part of the GAN model; this training thus trains only the generator model, and so ‘teaches’ the generator model how to ‘trick’ the discriminator.

According to certain embodiments, actual IMU gait representations are used as input, and, as part of the training cycle, the generator model may be trained with actual pairs of input and output (recorded from actual subjects). This ensures that while the output of the generator model becomes more and more ‘realistic’ to the viewer, or feasible, the output also maintains a direct connection to actual gait input e.g., as measured by the IMU. One or both models (of the two adversarial networks) may be exposed to other real life examples. For example, there are open libraries of BVH files for a range of activities, and IMU recordings of various activities may be available which may lack matched MoCap outputs. This provides the generative model with real IMU gait representations that are not part of the ground truth as input seeds in the GAN training operation, and the adversarial discriminator model typically learns random MoCap representations taken from other sources e.g. as described in the following http link: http://mocap.cs.cmu.edu/

Hence, rather than two operations, the following three operations may be used herein, for simultaneous training e.g., in operation 20:
t-1: A training operation for the discriminative model, which may comprise a conventional GAN training operation for discriminative models except that the synthetic samples of MoCap data are typically generated using real IMU inputs recorded by an IMU worn by a human, rather than random seeds, and/or
t-2: A training operation for the generative model, which may use procedures of conventional GAN training of the generative model, and/or
t-3: A training operation in which the generative model is trained in a “classic” way, with ground truth, as in the naive approach training operations.

Then, to use the adversarial nets, once trained, for prediction (e.g. in operation 40), the model as trained in operation 20 is applied to an input (e.g. to the representation produced in operation 30).

The cyclic convolutional layer may comprise the layer described in co-pending US application filed Oct. 13, 2021, USSN 17/500,744 - hereby incorporated herein by reference. This neural network layer may be implemented in both of the two adversarial networks for processing inputs to the layer, which may be based on a repetitive motion representation (which may be generated eg. by segmenting data representing a repetitive motion). A “cyclic convolutional layer” may be similar to a conventional 1-dimensional convolutional layer with padding, where the padding is typically as follows:

When padding, the input data is concatenated with a copy of the complementary edges on each side for a required length . The input to the cyclic convolutional layer typically comprises a N x M matrix of scalars, where one dimension, say the first, corresponds to time (thus may for example have a length of 100 units e.g. as described herein) thereby to define a first temporal end and second temporal end of the matrix. The other dimension corresponds to channels (e.g. x ,y , z displacement of the subject, for the initial or top layer). The required length is then the size of the kernel once (size of kernel-1)//2)) rows of the matrix been cropped from the second or later temporal end of the input matrix and (kernel//2)), rows of the matrix have been cropped from the first or earlier temporal end of the input matrix.

This cyclic convolutional layer typically characterizes the architecture of the generative and/or discriminative models and renders the model invariant to the starting point of the repetitive motion representation (e.g. phase 0 in standardization of the IMU input - the x component of the training set for the model training). Convolutional layers are affected by the boundaries of the previous layer; whereas the cyclic convolutional layer typically equally processes the data as the data could be rotated/shifted by any index The data may be rotated/shifted by any amount of units, and the output (of that data input with a cyclic convolutional layer) would remain the same other than rotation; when the last unit is rotated/shifted forward it reverts or goes back to the beginning of the matrix. Thus the edges do not affect the outcome.

In padding, e.g., as described, the input data may be concatenated to a copy of the complementary edges on each side for the required length (which typically depends on the convolutional layer’s kernel e.g., as described above) For example, it may be desired to use a cyclic convolutional layer with a kernel whose size is 10 units, and to apply that layer or kernel on or to an input of shape 100X6 (e.g., the standard shape of the input of the model, which is the standardized repetitive motion data from the IMU). The valid output of a 1-dimensional convolution of such a kernel with such an input may have a size or length of 91, since 91 indexes can be fully covered with such a kernel. As the data is repetitive, it may be considered cyclic, and thus the effect of the convolution may be defined as the signal repeats itself in its edges. Therefore, a piece of the beginning of the input data may be added at the end of the data, and a piece of the end of the input data at the beginning of the input data. In this example, 4 units (=(kernel-1)//2)), say, may be cropped from the later end of the data matrix and 5 units (=kernel)//2)), say, may be cropped from the earlier end of the matrix, which, in turn, produces an valid output whose length (e.g. 100), is the same as the input of the length.

It is appreciated that BVH files are mentioned herein merely by way of example; other alternatives may be employed such as FBX files.

When creating BVH (say) files, e.g., for operation 10d and/or for operation 40, any suitable structure described herein may be used. The BVH files’ structure may be produced, e.g. explicitly, from the output of any system described herein, such as but not limited to a gold-standard system, such as, for example, Xsens. The format e.g., for the y component of the pairs produced in operation 10d which may be used to train the model in operation 20, may be the MoCap’s standard representation of locomotion described elsewhere herein.

The MoCap standard locomotion representation (aka motion capture standard representation which may be generated by segmenting motion capture data) which may, for example, be used in operation/s 20 and/or 40, may include an output of a gold-standard motion capture system, such as but not limited to Xsens which is one example of an existing motion capture system where motion capture includes any process for recording the movement of objects or people typically by deploying a sensor associated with each body portion or joint whose trajectory is to be measured. Motion capture systems may generate BVH files, or may use other formats.

The following terms may be construed either in accordance with any definition thereof appearing in the prior art literature or in accordance with the specification, or to include in their respective scopes, the following:

The term MoCap measurement or motion capture data or MoCap data is used herein to include an output of a gold-standard MoCap system such as but not limited to Xsens, and MOCAP traditionally refers to the gold-standard motion capture system such as, for example, Xsens.

The term “MoCap-like outputs” is intended to include outputs, e.g. adversarial-network generated motion representations or MoCap approximations or MoCap predictions, generated by systems shown and described herein using adversarial networks as described herein; such outputs may include the output of the model generated in operation 20, given input recorded in operation 30. The term “MoCap” is also sometimes used herein to refer to the outputs of the system herein, e.g. to predictions or approximations of the MoCap data which would have been generated by gold-standard MoCap systems.

IMU gait cycle representation is used herein to refer to representations generated in operation 10d and/or 30c

Kinematics is used herein to include joint angle trajectory/ies which may for example be represented in a BVH file. The term kinematic signal may then be used to refer to contents of the BVH file.

The term raw data is used herein to refer to IMU data or MoCap data (BVH) as is, rather than a processed version of such data generated by operations 30b — 30c or 10c — 10d (e.g., cycle representation, or segmentation).

The processed version of such data may be referred to as a repetitive motion representation or as a “standard representation of repetitive pattern” or as a (MoCap or IMU) “repetition representation”.

The term segmentation (e.g. of a motion record) is used herein to refer to the process of dividing the measurement or motion record into repetitions e.g., strides (if the motion is a walking/running motion e.g.). For example, if a person monitored by Xsens walks 7 strides (if each of his 2 feet strides 7 times)), 7 segments may be provided. A single segment corresponds to a single repetition (e.g., gait cycle)

The term standardized data is used herein to refer to data which has undergone segmentation eg., in operations 10c or 30b.

The following terms may be interchanged herein: repetition, repetitive pattern, pattern repetitive motion, and are intended to include a motion by body part/s, which is repeated over and over, such as but not limited to a two-step gait cycle, an exercise, the act of ascending two stairs, leading with one foot for the first of the stairs, and with the other foot for the second of the stairs, and so forth. Typically, repetitioning is from the perspective of the IMU measurement, though, most typically, the repetitioning occurs in both the IMU and MoCap measurements simultaneously.

It is appreciated that a repetitive IMU pattern may comprise the input for operations 20 and/or 40, whereas repetitive segments of the MoCap may comprise labels for operation 20.

The terms phases or units are used herein to refer to multiple (say, 100) portions subdividing the duration of the repetitions. According to certain embodiments, there are, say, 100 uniform units dividing the duration (which is typically of the order of 1 second) of the repetitions, for standardization of the data. It is appreciated that if the repetition duration is ~ 1 second, and the sensors’ sampling rate is 100 hz, yielding 100 samples per second, it is typically not valuable to divide each repetition into more than 100 units.

X and y are the 2 simultaneous measurements collected in operation 10a - x from the IMU and y from the gold-standard MoCap system. Specifically:
X = Input = sample = IMU repetitive pattern representation aka IMU repetition representation aka IMU repetitive motion representation
Y = Label = output = MoCap’s standard representation of locomotion aka MoCap measurement;
typically comprises the segments in accordance with the IMU repetitions. Y is generated by the gold standard MoCap system e.g. Xsens.

According to certain embodiments, the model learns y from x and outputs MoCap-like data. The ground truth sample comprises multiple repetitive motion representations and labels; the ground truth dataset typically comprises both Input, e.g. samples of repetitive motion representation, and Labels e.g., corresponding MoCap outputs, which may comprise repetitive human locomotion representation, and may include BVH kinematics describing motion which occurred in the time interval of the IMU segments. According to certain embodiments, actual IMU gait representations (recorded from a human in motion who is wearing an IMU) are used as input, for training the generator model with actual pairs of input and output. Typically, each ML model inputs an IMU gait representation and outputs a MoCap standard locomotion representation, aka y.

It is appreciated that operations 10-20 (of the offline training phase) are applied only on the training set, whereas online operations 30-40 (of the prediction phase) are typically applied on demand/online. Thus operations 10, 30 have similarities, however, operation 10’s input includes data from the IMU and data (e.g. BVH file) from the gold-standard MoCap system, whereas operation 30’s input typically includes data from the IMU only. Thus, typically, synchronizing of two data sources occurs in operation 10, but not in operation 30. Operations 10c and 30b both include segmenting repetitions (time intervals) based on the IMU data (only).

It is appreciated that operations 10d and 30c are similar, except that in operation 10d the time intervals are applied to, or on, the MoCap data to yield the y components (repetitions), and these are then divided into multiple (e.g. c) time units. Operation 10d may then proceed in accordance with the method of operation 30c, and both may yield an x component which comprises a standardized form of the IMU data.

If, as in many contemplated use-cases, the input or x-component includes but a single IMU measurement, operation 30c is performed only once. If plural positions are collected, each is handled individually (each has its own ground truth e.g.) and operation 30c is performed once for each model trained.

If it is desired to estimate kinematics from two IMU measurements (from the two front/thigh pants pockets, for instance), all three measurements (2 IMUs and 1 MoCap measurement) are typically synchronized (as per operation 10b e.g.) to yield the following training set for the model: ((x1, x2), y). Segmentation operation 10c may be performed using either the left or right IMU data, or both Y is the MoCap data component as before X1 is the IMU component from the left pocket after applying the segmentation and standardization of operation 10d, and x2 is the right IMU component, also after applying the segmentation and standardization of operation 10d.

There is open source software such as blender (https://www.blender.org/) which derive animations of a human’s (or other subject’s) gait from standard formats such as BVH. According to embodiments herein, the BVH input to such systems need not be produced by complex hardware provided by a MoCap system and, instead, may be provided using only a cell phone whose IMU measures a gait cycle, e.g., using the flow of FIG. 4a.

Representation of motions of a person’s body portions, e.g., in BVH format, or animations derived therefrom, may be used as a vital sign of an end-user. Since the IMU is on-board a communication device e.g. smartphone, embodiments herein may facilitate tracking patients, elders, or other at-risk persons, even when unattended, in their home environment.

Representation of motions of a person’s body portions, eg. in BVH format, or animations derived therefrom, may also be used to facilitate ambulation rehabilitation, in which patients struggle to recover walking ability, following injuries, strokes, surgeries, etc. This provides a convenient, cost-effective, valuable tool for a physical therapist to track a patient’s walk between the patient’s relatively infrequent physical meetings to a clinic.

According to an embodiment, animation may be provided to a clinician or caregiver or therapist to enable that caregiver to observe the vital sign (or patient’s ambulation efforts) in a most intuitive and natural way, similar to actually seeing the at-risk person or patient.

Significant data to be flagged (e.g. representations of motions which merit triggering an alert regarding a given at-risk person) may be extracted from accumulations of the at-risk person’s gait data using any suitable known methods such as, for example, trend analysis and/or anomaly detection and/or pattern detection e.g. as described here:

https://www.researchgate.net/publication/329033123_Detecting_Irregular_Patterns_in_IoT_Stre aming_Data_for_Fall_Detection or here: https://rush-motion.com/

A smartphone, which may for example be in the end-user’s pocket, can record IMU data, typically representing a gait cycle, also in the background, when the end-user is not even aware of the measurements, thus reflecting the end-user’s most spontaneous and authentic behaviour.

Representation of motions of a person’s body portions, eg. in BVH format, or animations derived therefrom, may also be used for military, entertainment (such as filmmaking and video game development), sports, robotic and medical use-cases, including validation of computer vision.

Typically, human motion is recorded and the representations thereof may be used, say, to animate digital 2D or 3D avatars.

According to an embodiment of the present invention, a smartphone may be used to record motions of a human carrying or wearing the smartphone, without any need to engage with the smartphone camera, since the smartphone’s IMU records the inputs to the motion capture process e.g as described herein. Once body motions have thus been conveniently recorded, typically using only a smartphone, the recorded motion may be used to animate avatars in video products such as films, games and infotainment, e.g., by exporting the motions into animation software such as blender (https://www.blender.org/)

It is appreciated that some of the operations of FIG. 4a may be performed only once, and others, or even all operations, may be performed any suitable number of times e.g., n times, with the IMU device deployed in n bodily positions respectively (e.g., perhaps in two bodily positions - attached to right foot, and attached to left foot). It is appreciated that rather than repeating certain operations sequentially, with the IMU deployed in different bodily positions each time, n IMUs may be used, which may be deployed in n respective bodily positions.

It is appreciated that typically, plural joints’ kinematics are measured with the gold standard MoCap system, e.g., all joints. Therefore, the flow may be performed n times, for each of n body positions, if it is desired to generate n pairs of adversarial networks which extract the general kinematics regarding plural body portions, based on any one of the n body positions respectively.

For example, the gold standard MoCap system may deploy IMU devices on each of 12 joints. Potentially, 12 pairs of adversarial networks may be generated by running the flow of FIG. 4a, 12 times. The first pair will be useful in extracting general kinematics regarding all 12 body portions, based on a single IMU deployed at a first of the 12 body positions, say a pants pocket. The second pair of adversarial networks will be useful in extracting general kinematics regarding all 12 body portions, based on a single IMU deployed at a second of the 12 body positions, and the last pair of adversarial networks will be useful in extracting general kinematics regarding all 12 body portions, based on a single IMU deployed at the last of the 12 body positions. Thus, plural bodily positions may be measured in parallel, or may be collected individually e.g., if it is desired to, ex post facto, add more bodily positions by collecting a new data set.

If several ground truth datasets have been collected, and several ML models trained respectively from the several ground truth datasets, the outputs of the models may, if desired, be combined.

According to some embodiments, a model from among several models trained may be selected by recognizing the position from which the IMU measurement was taken, and selecting the model which was trained to predict general kinematics based on that specific bodily position. And/or, the user may be prompted, via a user interface, to identify the position from which the IMU measurement was taken. Example: it is desired to generate joint kinematics for 7 lower body joints - lower back, left hip, left knee, left ankle; and same 3 joints also for right.

Thus, all or any subset of the operations in FIG. 4a may be repeated 7 times, once for each of the 7 bodily positions of the input (of the IMU data). For example, if it is desired to train models for estimating the 7 lower body joints using data collected from only the right pants pocket, or only the left hip pocket, one model may be trained for the right pocket, and one for the left pocket. The output of each of the models includes joint kinematics for all 7 joints.

### Example (Aka “Appendix A”)

An example BVH file, generated using a MoCap system (Xsens), is as follows; the header of the file appears in bold, and the data appears in italics.

The BVH file below includes 5 initial frames from among 108368 frames in total, which were produced by a MoCap system that recorded a subject who was walking naturally

The skeleton joints comprise hips, 4 upper spinal joints (chest#), neck, head, 4 joints for each arm, and 4 joints for each leg, totalling 23 joints.

HIERARCHY
ROOT Hips
{
OFFSET 0.000000 0.000000 0.000000
CHANNELS 6 Xposition Yposition Zposition Yrotation Xrotation Zrotation
JOINT Chest
{
OFFSET 0.000000 0.092923 -0.010325
CHANNELS 3 Yrotation Xrotation Zrotation
JOINT Chest2
{
OFFSET 0.000000 0.102448 0.000000
CHANNELS 3 Yrotation Xrotation Zrotation
JOINT Chest3
{
OFFSET 0.000000 0.093543 -0.000075
CHANNELS 3 Yrotation Xrotation Zrotation
JOINT Chest4
{
OFFSET 0.000000 0.093438 -0.000075
CHANNELS 3 Yrotation Xrotation Zrotation
JOINT Neck
{
OFFSET 0.000000 0.130922 0.000000
CHANNELS 3 Yrotation Xrotation Zrotation
JOINT Head
{
OFFSET 0.000000 0.087010 0.000162
CHANNELS 3 Yrotation Xrotation Zrotation
End Site
{
OFFSET 0.000000 0.161413 0.000022
}
}
}
JOINT RightCollar
{
OFFSET -0.028643 0.073433 0.000000
CHANNELS 3 Yrotation Xrotation Zrotation
JOINT RightShoulder
{
OFFSET -0.135041 0.000000 0.000000
CHANNELS 3 Yrotation Xrotation Zrotation
JOINT RightElbow
{
OFFSET -0.289825 0.000000 0.000000
CHANNELS 3 Yrotation Xrotation Zrotation
JOINT RightWrist
{
OFFSET -0.236769 0.000000 0.000000
CHANNELS 3 Yrotation Xrotation Zrotation
End Site
{
OFFSET -0.176562 0.000000 0.000000
}
}
}
}
}
JOINT LeftCollar
{
OFFSET 0.028643 0.073433 0.000000
CHANNELS 3 Yrotation Xrotation Zrotation
JOINT LeftShoulder
{
OFFSET 0.135041 0.000000 0.000000
CHANNELS 3 Yrotation Xrotation Zrotation
JOINT LeftElbow
{
OFFSET 0.289825 0.000000 0.000000
CHANNELS 3 Yrotation Xrotation Zrotation
JOINT LeftWrist
{
OFFSET 0.236769 0.000000 0.000000
CHANNELS 3 Yrotation Xrotation Zrotation
End Site
{
OFFSET 0.176562 0.000000 0.000000
}
}
} } } }
}
JOINT RightHip
{
OFFSET -0.076704 0.000195 -0.000022
CHANNELS 3 Yrotation Xrotation Zrotation
JOINT RightKnee
{
OFFSET 0.000000 -0.397684 -0.000018
CHANNELS 3 Yrotation Xrotation Zrotation
JOINT RightAnkle
{
OFFSET 0.000000 -0.387529 -0.000030
CHANNELS 3 Yrotation Xrotation Zrotation
JOINT RightToe
{
OFFSET 0.000000 -0.071252 0.162445
CHANNELS 3 Yrotation Xrotation Zrotation
End Site
{
OFFSET 0.000000 -0.015311 0.067101
}
}
}
}
}
JOINT LeftHip
{
OFFSET 0.076704 0.000174 -0.000019
CHANNELS 3 Yrotation Xrotation Zrotation
JOINT LeftKnee
{
OFFSET 0.000000 -0.397684 -0.000018
CHANNELS 3 Yrotation Xrotation Zrotation
JOINT LeftAnkle
{
OFFSET 0.000000 -0.387529 -0.000030
CHANNELS 3 Yrotation Xrotation Zrotation
JOINT LeftToe
{
OFFSET 0.000000 -0.071252 0.162445
CHANNELS 3 Yrotation Xrotation Zrotation
End Site
{
OFFSET 0.000000 -0.015311 0.067101
}
}
}
}
}
}
MOTION
Frames: 108368
Frame Time: 0.010000
0.000000 0.871034 0.000000 0.000000 6.340192 0.000000 0.000000 -6.340192 0.000000
0.000000 -5.826342 0.000000 0.000000 -0.005910 0.000000 0.000000 5.832252 0.000000
0.000000 12.610213 0.000000 0.000000 -9.243752 0.000000 0.000000 0.000000 0.000000
0.000000 0.000000 0.000000 2.309063 0.000000 0.000000 -2.309063 0.000000 0.000000
0.000000 0.000000 0.000000 0.000000 0.000000 0.000000 -2.309063 0.000000 0.000000
2.309063 0.000000 0.000000 0.000000 -3.591006 0.000000 0.000000 1.872585 0.000000
0.000000 -4.621771 0.000000 0.000000 0.000000 0.000000 0.000000 -3.587706 0.000000
0.000000 1.905768 0.000000 0.000000 -4.658254 0.000000 0.000000 0.000000 0.000000
0.036464 0.876554 0.248925 -12.889058 8.274984 -0.259903 0.000000 -2.8425413 -0.000000
0.000000 -7.089694 0.000000 0.000000 -1.269263 0.000000 -0.000000 4.884738 -0.000000
0.007768 12.401612 0.498138 0.101583 -12.401204 -0.508330 0.000000 -0.000000 0.000000
0.000000 -0.000000 0.000000 0.000000 -0.000000 0.000000 0.000000 -0.000000 0.000000
0.000000 -0.000000 0.000000 0.000000 -0.000000 0.000000 0.000000 -0.000000 0.000000
0.000000 -0.000000 0.000000 0.360294 -6.739821 1.092447 0.490163 0.157112 0.461171
1.407038 -6.783032 -4.409937 0.000000 -0.000000 0.000000 6.342363 -5.849298 -0.499978
0.154172 1.419916 0.206654 5.379639 -8.962582 -0.695187 0.000000 -0.000000 0.000000
0.036573 0.876244 0.249079 -12.874323 8.249739 -0.265628 -0.000000 -2.842543 0.000000 -
0.000000 -7.089694 0.000000 0.000000 -1.269263 0.000000 -0.000000 4.884738 -0.000000
0.009376 12.401990 0.503850 0.101195 -12.401588 -0.513826 0.000000 -0.000000 0.000000
0.000000 -0.000000 0.000000 0.000000 -0.000000 0.000000 0.000000 -0.000000 0.000000
0.000000 -0.000000 0.000000 0.000000 -0.000000 0.000000 0.000000 -0.000000 0.000000
0.000000 -0.000000 0.000000 0.340681 -6.708810 1.064216 0.506360 0.159445 0.537494
1.406779 -6.782899 -4.455205 0.000000 -0.000000 0.000000 6.312212 -5.817796 -0.510127
0.176316 1.414183 0.244751 5.376083 -8.953746 -0.711676 0.000000 0.000000 0.000000
0.036680 0.875943 0.249231 -12.859704 8.224704 -0.271248 -0.000000 -2.842543 0.000000 -
0.000000 -7.089694 0.000000 0. 000000 -1.269263 0.000000 -0.000000 4.884738 -0.000000
0.010963 12.402364 0.509485 0.100814 -12.401966 -0.519247 0.000000 -0.000000 0.000000
0.000000 -0.000000 0.000000 0.000000 -0.000000 0.000000 0.000000 -0.000000 0.000000
0.000000 -0.000000 0.000000 0.000000 -0.000000 0.000000 0.000000 -0.000000 0.000000
0.000000 -0.000000 0.000000 0.321338 -6.678069 1.036285 0.522294 0.161837 0.612567
1.406571 -6. 782736 -4.499726 0.000000 0.000000 0.000000 6.282282 -5. 786545 -0.520204
0.198248 1.408560 0.282302 5.372609 -8.944933 -0.727924 0.000000 0.000000 0.000000
0.036779 0.875674 0.249376 -12.845554 8.200507 -0.276431 -0.000000 -2.842543 0.000000
0.000000 -7.089694 0.000000 0.000000 -1.269263 0.000000 -0.000000 4.884738 -0.000000
0.012466 12.402722 0.514813 0.100450 -12.402328 -0.524372 0.000000 -0.000000 0.000000
0.000000 -0.000000 0.000000 0.000000 -0.000000 0.000000 0.000000 -0.000000 0.000000
0.000000 -0.000000 0.000000 0.000000 -0.000000 0.000000 0.000000 -0.000000 0.000000
0.000000 -0.000000 0.000000 0.303069 -6.648407 1.009590 0.537141 0.164550 0.682643
1.406584 -6.782469 -4.541262 0.000000 -0.000000 0.000000 6.253222 -5.756308 -0.529928
0.219340 1.403415 0.317682 5.369473 -8.936206 -0. 743209 0.000000 0.000000 0.000000

It is appreciated that terminology such as “mandatory”, “required”, “need” and “must” refer to implementation choices made within the context of a particular implementation or application described herewithin for clarity, and are not intended to be limiting, since, in an alternative implementation, the same elements might be defined as not mandatory and not required, or might even be eliminated altogether.

Components described herein as software may, alternatively, be implemented wholly or partly in hardware and/or firmware, if desired, using conventional techniques, and vice-versa. Each module or component or processor may be centralized in a single physical location or physical device, or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order, including simultaneous performance of suitable groups of operations, as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order i.e. not necessarily as shown, including performing various operations in parallel or concurrently rather than sequentially as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform e.g. in software any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations, or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented e.g., by one or more processors. The invention shown and described herein may include (a) using a computerized method to identify a solution to any of the problems or for any of the objectives described herein, the solution optionally including at least one of a decision, an action, a product, a service, or any other information described herein that impacts, in a positive manner, a problem or objectives described herein; and (b) outputting the solution

The system may,, if desired be implemented as a network- e.g. web-based system employing software, computers, routers and telecommunications equipment, as appropriate.

Any suitable deployment may be employed to provide functionalities e.g., software functionalities shown and described herein. For example, a server may store certain applications, for download to clients, which are executed at the client side, the server side serving only as a storehouse. Any or all functionalities e.g., software functionalities shown and described herein, may be deployed in a cloud environment. Clients e.g, mobile communication devices such as smartphones, may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are,, if they so desire able to modify the device to obtain the structure or function.

Any “if -then” logic described herein is intended to include embodiments in which a processor is programmed to repeatedly determine whether condition x, which is sometimes true and sometimes false, is currently true or false, and to perform y each time x is determined to be true, thereby to yield a processor which performs y at least once, typically on an “if and only if” basis, e.g. triggered only by determinations that x is true, and never by determinations that x is false.

Any determination of a state or condition described herein, and/or other data generated herein, may be harnessed for any suitable technical effect. For example, the determination may be transmitted or fed to any suitable hardware, firmware or software module, which is known or which is described herein to have capabilities to perform a technical operation responsive to the state or condition. The technical operation may,, for example comprise changing the state or condition, or may more generally cause any outcome which is technically advantageous given the state or condition or data, and/or may prevent at least one outcome which is disadvantageous given the state or condition or data. Alternatively or in addition, an alert may be provided to an appropriate human operator or to an appropriate external system.

Features of the present invention, including operations which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art, and particularly, although not limited to, those described in the Background section, or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g., as illustrated or described herein.

Devices, apparatus or systems shown coupled in any of the drawings may in fact be integrated into a single platform in certain embodiments. or may be coupled via any appropriate wired or wireless coupling, such as but not limited to optical fiber, Ethernet, Wireless LAN, HomePNA, power line communication, cell phone, Smart Phone (e.g. iPhone), tablet, laptop, PDA, Blackberry GPRS, satellite including GPS, or other mobile delivery. It is appreciated that in the description and drawings shown and described herein, functionalities described or illustrated as systems and sub-units thereof can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation, and is not intended to be limiting.

Any suitable communication may be employed between separate units herein e.g, wired data communication and/or in short-range radio communication with sensors such as cameras e.g. via WiFi, Bluetooth, or Zigbee.

It is appreciated that implementation via a cellular app as described herein is but an example and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node).

Any operation or characteristic described herein may be performed by another actor outside the scope of the patent application and the description is intended to include apparatus, whether hardware, firmware or software, which is configured to perform, enable or facilitate that operation or to enable, facilitate or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry, including any such computer microprocessor/s, as well as in firmware or in hardware, or any combination thereof.

It is appreciated that elements illustrated in more than one drawing, and/or elements in the written description, may still be combined into a single embodiment, except if otherwise specifically clarified herewithin. Any of the systems shown and described herein may be used to implement or may be combined with, any of the operations or methods shown and described herein.

It is appreciated that any features, properties, logic, modules, blocks, operations or functionalities described herein, which are, for clarity, described in the context of separate embodiments, may also be provided in combination in a single embodiment, except where the specification or general knowledge specifically indicates that certain teachings are mutually contradictory, and cannot be combined. Any of the systems shown and described herein may be used to implement, or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element e.g., operation described herein, may have all characteristics and attributes described or illustrated herein, or, according to other embodiments, may have any subset of the characteristics or attributes described herein.

It is appreciated that apps constructed to implement embodiments herein may include a cell app, mobile app, computer app, or any other application software. Any application may be bundled with a computer and its system software, or published separately. The term “phone” and similar used herein is not intended to be limiting, and may be replaced or augmented by any device having a processor, such as but not limited to a mobile telephone, or also set-top-box, TV, remote desktop computer, game console, tablet, mobile e.g. laptop, or other computer terminal, embedded remote unit, which may either be networked itself (may itself be a node in a conventional communication network e.g.) or may be conventionally tethered to a networked device (to a device which is a node in a conventional communication network, or is tethered directly or indirectly/ultimately to such a node). Thus, the computing device may even be disconnected from e.g., WiFi, Bluetooth, etc., but may be tethered directly or ultimately to a networked device.

1. A system for capturing motion of a moving body, the system including:
an IMU interface configured to receive IMU measurements from at least one IMU worn on the moving body; and
a hardware processor configured to derive from the IMU measurements, a motion capture approximation output including a trajectory, for each individual body portion from among B body portions, which describes the individual body portion’s motion during a single repetition (aka cycle) of a repetitive motion, thereby to provide a trajectory set including B body portion trajectories (aka “repetitive activity patterns”),
wherein the hardware processor uses generative adversarial networks to derive the trajectory set from the IMU measurements,
the generative adversarial networks including:
a first network trained to determine physical feasibility of at least one candidate body portion trajectory for at least one specific body portion, from among a multiplicity of candidate body portion trajectories for said specific body portion; and
a second network trained to determine how well at least one candidate body portion trajectory, from among the multiplicity of candidate body portion trajectories, fits the IMU measurements.

2. The system of claim 1 wherein the B body portion trajectories are represented in BVH format.

3. The system of claim 1 wherein at least one IMU comprises a single IMU deployed at a single body location.

4. The system of claim 1 wherein at least one IMU comprises plural IMU’s deployed at plural respective body locations.

5. The system of claim 1 wherein the first network includes a discriminative network trained to determine how feasible is/are candidate body portion trajectory/ies from among a multiplicity of candidate body portion trajectories, and wherein the second network includes a generative network.

6. The system of claim 5 wherein the generative network is trained to predict body portion trajectory/ies, by identifying candidate body portion trajectory/ies which fit/s the IMU measurements, from among candidate body portion trajectory/ies considered feasible by the discriminative network.

7. The system of claim 1 wherein said IMU measurements comprise acceleration measurements and orientation measurements.

8. The system of claim 1 wherein the IMU comprises at least one of: an accelerometer; a gyroscope, a magnetometer.

9. The system of claim 1 wherein said body part includes at least one of: a joint, a limb portion, a limb.

10. The system of claim 1 and wherein the hardware processor is also configured for segmentation of IMU measurements of at least one body portion’s motion, into repetitions, aka cycles.

11. The system of claim 1 wherein the trajectory of at least one body portion from among B body portions, which describes that body portion’s motion during a single repetition, is agnostic to the IMU’s orientation.

12. The system of claim 1 wherein the IMU is tri-axial.

13. The system of claim 1 wherein training data to train at least one of the first and second networks includes IMU measurements provided by said at least one IMU during at least one time interval, wherein the IMU measurements are synchronized to gold-standard motion capture measurements made during said at least one time interval.

14. The system of claim 1 wherein said motion capture approximation output, rather than motion capture data, is fed to at least one apparatus which utilizes motion capture data.

15. The system of claim 14 wherein said apparatus comprises software which generates at least one animation of an avatar from motion capture data representing a motion, performed by a human, which the avatar is to perform.

16. The system of claim 14 wherein said apparatus comprises a device for tracking at-risk persons including using motion capture data as a vital sign indicative of wellbeing of the at-risk person.

17. The system of claim 14 wherein said apparatus comprises an ambulation rehabilitation tool which uses motion capture data to track a patient’s ambulation between the patient’s physical meetings with a clinician.

18. The system of claim 14 wherein said apparatus comprises trend analysis and/or anomaly detection and/or pattern detection software which detects trends and/or anomalies and/or patterns in motion capture data.

19. The system of claim 1 wherein the IMU is co-located with a first moving body portion having a first trajectory of motion, and wherein the system uses readings generated by the IMU to approximate motion capture data regarding motion of at least one second moving body portion having a second trajectory of motion which differs from said first trajectory of motion.

20. The system of claim 19 wherein said second portion’s motion affects said first body portion’s motion.

21. The system of claim 19 wherein said second portion’s motion is affected by said first body portion’s motion.

22. A method for capturing motion of a moving body, the method including:
providing an IMU interface configured to receive IMU measurements from at least one IMU worn on the moving body; and
deriving, from the IMU measurements, using a hardware processor, a motion capture approximation output including a trajectory, for each individual body portion from among B body portions, which describes the individual body portion’s motion during a single repetition (aka cycle) of a repetitive motion, thereby to provide a trajectory set including B body portion trajectories (aka “repetitive activity patterns”),
wherein the hardware processor uses generative adversarial networks to derive the trajectory set from the IMU measurements, the generative adversarial networks including:
a first network trained to determine physical feasibility of at least one candidate body portion trajectory for at least one specific body portion, from among a multiplicity of candidate body portion trajectories for said specific body portion; and
a second network trained to determine how well at least one candidate body portion trajectory, from among the multiplicity of candidate body portion trajectories, fits the IMU measurements.

23. A computer program product, comprising a non-transitory tangible computer readable medium having computer readable program code embodied therein, said computer readable program code adapted to be executed to implement a method for capturing motion of a moving body, the method including:
providing an IMU interface configured to receive IMU measurements from at least one IMU worn on the moving body; and
deriving, from the IMU measurements, using a hardware processor, a motion capture approximation output including a trajectory, for each individual body portion from among B body portions, which describes the individual body portion’s motion during a single repetition (aka cycle) of a repetitive motion, thereby to provide a trajectory set including B body portion trajectories (aka “repetitive activity patterns”),
wherein the hardware processor uses generative adversarial networks to derive the trajectory set from the IMU measurements, the generative adversarial networks including:
a first network trained to determine physical feasibility of at least one candidate body portion trajectory for at least one specific body portion, from among a multiplicity of candidate body portion trajectories for said specific body portion; and
a second network trained to determine how well at least one candidate body portion trajectory, from among the multiplicity of candidate body portion trajectories, fits the IMU measurements.
